(19) United States
(12) Patent Application Publication
(10) Pub. No.: US 2024/0294565 A1
(43) Pub. Date: Sep. 5, 2024

(54) UNNATURAL CONFIGURATION NUCLEOTIDE PRODRUG COMPOUNDS

(71) Applicant: Ligand Pharmaceuticals Incorporated, San Diego, CA (US)

(72) Inventor: Lin Zhi, Austin, TX (US)

(21) Appl. No.: 18/561,681

(22) PCT Filed: May 10, 2022
(86) PCT No.: PCT/US2022/028490

§ 371 Date: Nov. 16, 2023

Related U.S. Application Data
(60) Provisional application No. 63/189,402, filed on May 17, 2021.
(60) Provisional application No. 63/256,419, filed on Oct. 15, 2021.

(51) Int. Cl. C07H 19/10; A61K 31/44; A61K 31/7072; C07H 19/11

(52) U.S. Cl. CPC C07H 19/10; A61K 31/44; A61K 31/7072; C07H 19/11

(57) ABSTRACT

Provided herein am phosphoramidate and cyclophosphate prodrug compounds of unnatural configuration nucleotide, their preparation, and their uses, such as treating liver diseases or nonliver diseases via intervening in the molecular pathways in the liver.

### FIELD

The present disclosure relates to the field of chemistry and medicine. More specifically, the present disclosure relates to phosphoramidate and cyclophosphate compounds, their preparation and their uses. In some embodiments, such compounds are useful to selectively deliver certain unnatural configuration nucleotides to the liver.

### BACKGROUND

The following description of the background is provided to aid in understanding the invention, but is not admitted to be, or to describe, prior art to the invention.

Natural nucleos(t)ide compounds are essential molecular building blocks of life and many nucleos(t)ide analog compounds are widely used as antiviral and anticancer agents in the form of prodrugs to improve efficiency and safety to treat a disease. Most of the nucleos(t)ide analog compounds are structurally modified based on the natural nucleoside subunit structural configuration exist in DNA and RNA. Limited number of nucleos(t)ide analog compounds are derived from the mirror configuration (unnatural) of the nucleoside subunits of DNA and RNA.

The unnatural nucleos(t)ide chemistry space is less explored and may provide the opportunity to develop novel drugs. Novel prodrugs of the known or the to-be-discovered unnatural nucleos(t)ides may help to fight the existing or emerging viral diseases and/or cancer.

### SUMMARY

Novel prodrug compounds of unnatural nucleos(t)ides, their preparation and their uses are described. Some embodiments are related to novel phosphoramidate and cyclophosphate compounds that are absorbed in the intestine and taken up via the hepatic portal vein to the liver where the compounds provide a therapeutic benefit. Another aspect includes the use of the phosphoramidate and cyclophosphate compounds to treat diseases that benefit from enhanced drug distribution to the liver and like tissues and cells, including but not limited to hepatitis, cancer, liver fibrosis, fatty liver, and metabolic, and cardiovascular diseases where the liver is involved in the production and/or the homeostasis control of the biochemical end products, e.g. glucose, cholesterol, fatty acids, triglycerides, lipoproteins, and apolipoproteins. In another aspect, the phosphoramidate and cyclophosphate compounds are used to increase the pharmacological or clinical activity of certain unnatural nucleos(t)ide analog compounds. In another aspect, the phosphoramidate and cyclophosphate compounds are used to reduce potential side effects of certain unnatural nucleos(t)ide analog compounds, especially the side effects occurring outside the liver. In some embodiments, the phosphoramidate and cyclophosphate compounds are useful in the delivery of diagnostic imaging agents to the liver.

Some embodiments provided herein include a compound of Formula I, II, III, IV, V, VI, or VII:

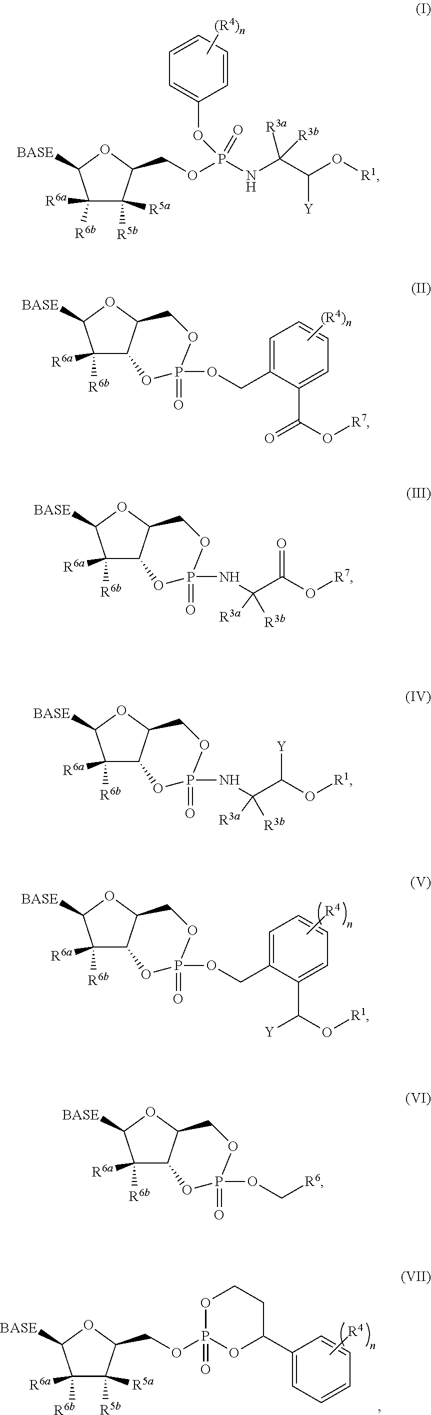

or a stereoisomer or a pharmaceutically acceptable salt thereof, wherein R¹, R², R^{3a}, R^{3b}, R⁴, R^{5a}, R^{5b}, R^{6a}, R^{6b}, R⁷, R⁸, n, Y, and Base have any of the values described herein.

Some embodiments relate to a pharmaceutical composition comprising any of the above compounds and a pharmaceutically acceptable excipient.

Some embodiments relate to a method of treating a disease, disorder or condition comprising administering an effective amount of any of the above compounds.

In some embodiments, the disease, disorder or condition is a disease, disorder or condition of the liver.

In some embodiments, the disease, disorder or condition is a non-liver disease, disorder or condition.

In some embodiments, the disease, disorder or condition is a metabolic, cardiovascular or hormonal disease in which the liver is involved in the production and/or the homeostasis control of the biochemical end products of the disease, disorder or condition.

In some embodiments, the disease, disorder or condition is selected from the group consisting of hepatitis, cancer, liver fibrosis, fatty liver, malaria, viral infection, parasitic infection, diabetes, hyperlipidemia, atherosclerosis, obesity, dyslipidemia, hyperglycemia and a hormonal condition.

In some embodiments, the non-liver disease, disorder or condition is a viral infection, cancer, or other disease in which the prodrug compounds enhance the distribution of an active drug to the target tissue or cell.

Some embodiments further comprise administering an effective amount of at least one additional therapeutic agent to the subject in need thereof.

Some embodiments relate to a method of delivering a diagnostic imaging agent to the liver of a subject in need thereof, comprising administering to the subject an effective amount of any of the above compounds.

In some embodiments, the subject is a mammal.

In some embodiments, the subject is human.

Some embodiments relate to a method of inhibiting viral replication in a cell comprising contacting the cell with any of the above compounds.

Some embodiments relate to a method of intervening in a molecular pathway or modulating a target in a cell comprising contacting the cell with any of the above compounds.

In some embodiments, the cell is in vivo.

In some embodiments, the cell is ex vivo.

In some embodiments, the cell is a hepatocyte.

In some embodiments, the cell is mammalian.

In some embodiments, the cell is human.

Some embodiments of the compounds, compositions, and methods provided herein include a pharmaceutical composition comprising any of the compounds provided herein and a pharmaceutically acceptable excipient.

Some embodiments of the compounds, compositions, and methods provided herein include a method of treating a disease or condition in the liver in a subject comprising administering an effective amount of any of the compounds provided herein to a subject in need thereof.

Some embodiments also include administering an effective amount of one or more additional therapeutic agents to the subject in need thereof.

In some embodiments, the subject is a mammal.

In some embodiments, the subject is a human.

Some embodiments also include the use of any one of the compounds provided herein in combination with an additional therapeutic agent.

Some embodiments of the compounds, compositions, and methods provided herein include any one of the compositions provided herein for use in the preparation of a medicament for treating a disease or condition in the liver or a disease or condition in which the physiological or pathogenic pathways involve the liver.

### DETAILED DESCRIPTION

Nucleoside monophosphates and monophosphonates are attractive drug targets as conversion to the active triphosphate is rapid and the initial rate limiting phosphorylation is not required. However, nucleoside monophosphates are limited as drug candidates as they are not efficiently transported to therapeutic target in cells. Thus, strategies have been developed to facilitate delivery of nucleoside monophosphates into cells. The unnatural nucleoside based drugs clevudine and telbivudine are approved to treat hepatitis B with limited use. Scheme 1 provides the activation mechanism of clevudine and telbivudine. The first phosphorylation is the rate-limiting step when the nucleosides reach the liver. The monophosphate of clevudine is known to undergo rapid phosphorylation to the active triphosphate form (e.g., See K. E. Squires, et al. Antimicrobial Agents and Chemotherapy, (2020), 64(9):1-13).

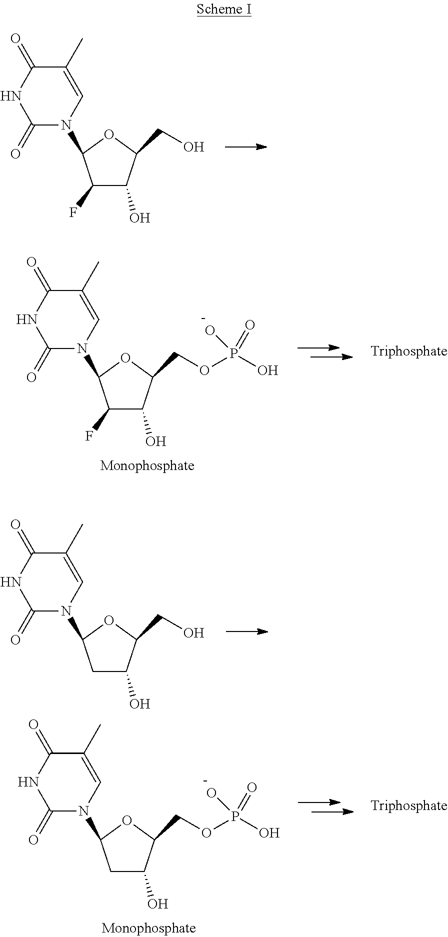

The present embodiments are directed to compositions and methods related to novel prodrug compounds, their preparation and their uses. In some embodiments, the novel phosphoramidate and cyclophosphate compounds facilitate delivery into cells of monophosphate therapeutic agents, such as nucleoside monophosphates. In some embodiments, the novel phosphoramidate and cyclophosphate compounds facilitate delivery of the unmasked monophosphate of the unnatural nucleosides into cells.

These phosphoramidate and cyclophosphate compounds and their stereoisomers and pharmaceutically acceptable salts are represented by Formula I, II, III, IV, V, VI and VII:

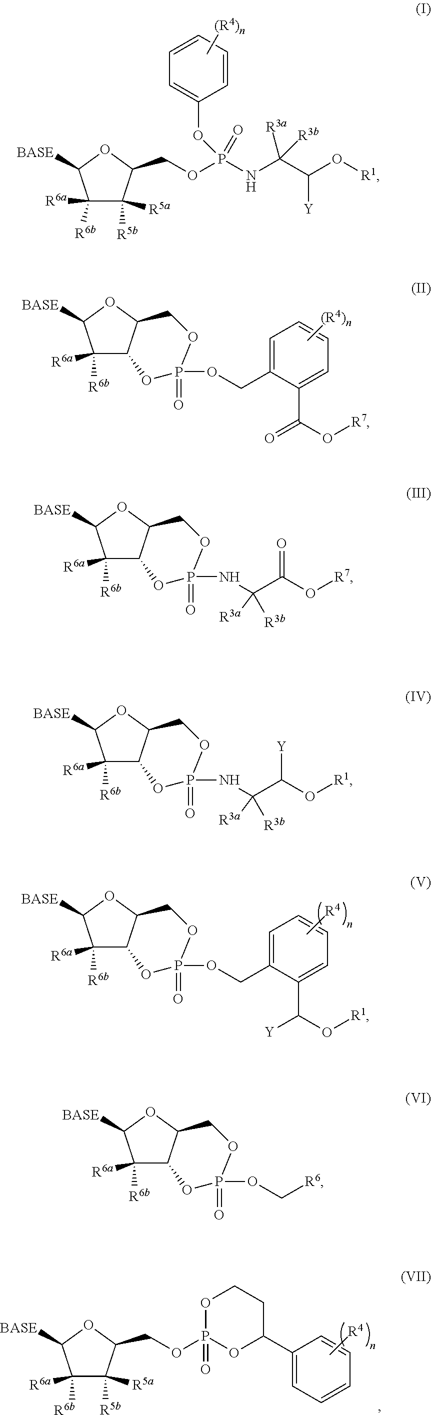

wherein R¹, R², R^{3a}, R^{3b}, R⁴, R^{5a}, R^{5b}, R^{6a}, R^{6b}, R⁷, R⁸, n, Y, and Base have any of the values described herein.

In some embodiments, Y is H or —OR²; R¹ and R² are independently H, an optionally substituted C₁-C₁₀ alkyl, an optionally substituted C₅-C₁₀ aryl, an optionally substituted C₁-C₆ alkyl-C(O)—, an optionally substituted five to ten membered heteroaryl, or an optionally substituted —(CH₂)ₓ—(C₅-C₁₀ aryl); or alternatively, R¹ and R² may be taken together with the atoms to which they are attached and intervening atoms to form a four to ten membered heterocycle optionally substituted with between one and four C₁-C₆ alkyl groups; R^{3a} and R^{3b} are independently H or an optionally substituted C₁-C₁₀ alkyl; or alternatively, R^{3a} and R^{3b} may be taken together with the atom to which they are attached form a three to seven membered ring; R⁴ is halo, an optionally substituted C₁-C₁₀ alkyl, or an optionally substituted C₁-C₆ alkyloxy; R^{5a}, R^{5b}, R^{6a}, and R^{6b} are independently selected from the group consisting of H, OR^{A}, halo, —CN, and an optionally substituted C₁-C₁₀ alkyl; R^{A} is selected from the group consisting of H, an optionally substituted C₁-C₆ acyl, an optionally substituted C₁-C₆ alkyloxycarbonyl; and an optionally substituted C₁-C₆ carbamoyl; R⁷ is H or an optionally substituted C₁-C₁₀ alkyl; R⁸ is an optionally substituted C₁-C₁₀ alkyl, an optionally substituted phenyl, or an optionally substituted benzyl; n is 0, 1, 2, or 3; x is 0, 1, or 2; and Base is a natural nucleoside base or a derivative or analog thereof.

In other embodiments, R¹ is a C₁-C₆ alkyl. In other embodiments, R¹ is methyl. In other embodiments, R¹ is ethyl. In other embodiments, R¹ is propyl. In other embodiments, R¹ is butyl. In other embodiments, R¹ is H. In other embodiments, aryl is phenyl. In other embodiments, x is 1. In some embodiments, Y is H. In other embodiments, Y is —OR². In other embodiments, R² is methyl. In other embodiments, R² is ethyl. In other embodiments, R² is propyl. In other embodiments, R² is butyl.

In some embodiments, R¹ and R² are taken together with the atoms to which they are attached and intervening atoms to form a four to ten membered heterocycle optionally substituted with between one and four C₁-C₆ alkyl groups. In some embodiments, R¹ and R² are taken together with the atoms to which they are attached and intervening atoms to form

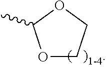

In other embodiments, R¹ and R² are taken together with the atoms to which they are attached and intervening atoms to form

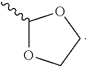

In other embodiments, R¹ and R² are taken together with the atoms to which they are attached and intervening atoms to form

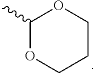

In some embodiments, R^{3a} and R^{3b} are both H. In other embodiments, R^{3a} is H and R^{3b} is methyl. In other embodiments, R^{5a} is H. In other embodiments, R^{5b} is —OR^{A}. In other embodiments, R^{A} is H. In other embodiments, R^{5b} is

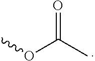

In other embodiments, R^{5b} is

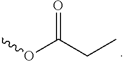

In some embodiments, R⁷ is C₁-C₆ alkyl. In other embodiments, R⁷ is methyl. In some embodiments, R⁷ is ethyl. In other embodiments, R⁷ is propyl. In other embodiments, R⁷ is i-Pr. In other embodiments, R⁷ is butyl. In other embodiments, R⁷ is i-butyl.

In some embodiments, R^{6a} is halo. In other embodiments, R is fluoro. In other embodiments, R^{6b} is H. In some embodiments, R⁴ is halo selected from fluoro and choro. In other embodiments, n is 1. In some embodiments, n is 2. In some embodiments, one R⁴ is fluoro and one R⁴ is chloro.

In some embodiments, R⁸ is selected from the group consisting of an optionally substituted C₁-C₁₅ alkyl, an optionally substituted aryl, an optionally substituted benzyl, an optionally substituted phenylethyl, and an optionally substituted phenylpropyl. In some embodiments, R⁸ is C₁-C₁₅ alkyl, in some embodiments, R⁸ is pentyl, in some embodiments, R⁸ is hexyl, in some embodiments, R⁸ is heptyl, in some embodiments, R⁸ is phenyl, in some embodiments R⁸ is fluorophenyl, in some embodiments, R⁸ is benzyl, in some embodiments, R⁸ is phenylethyl.

In some embodiments, Base is selected from the group consisting of

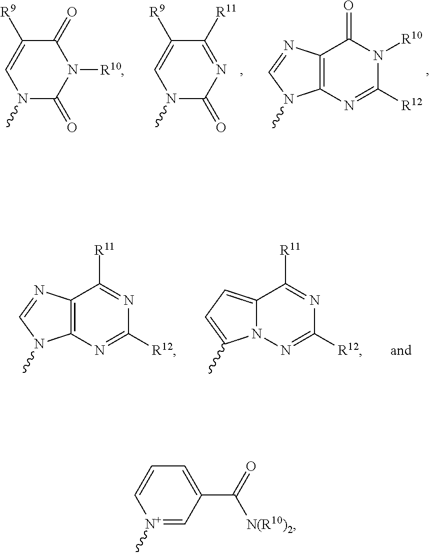

wherein: R⁹ is H, halo, —CD₃, or an optionally substituted C₁-C₁₀ alkyl; R¹⁰ is selected from the group consisting of H, an optionally substituted C₁-C₁₀ alkyl, an optionally substituted C₁-C₁₀ alkyl-OCH₂—, an optionally substituted C₁-C₁₀ alkyl-NHCH₂—, an optionally substituted C₁-C₁₀ acyl, an optionally substituted C₁-C₁₀ alkyl-OC(O)—, an optionally substituted (C₆₋₁₀ aryl)-CH₂OCH₂—, an optionally substituted (C₆₋₁₀ aryl)-OCH₂—, an optionally substituted (C₆₋₁₀ aryl)-C(O)—, and an optionally substituted (C₆₋₁₀ aryl)-OC(O)—; R¹¹ is selected from the group consisting of OH, NH₂, NHOH, an optionally substituted C₁-C₁₀ alkyloxy, an optionally substituted C₁-C₁₀ alkylamino, an optionally substituted C₁-C₁₀ acyloxy, an optionally substituted C₁-C₁₀ acylamino, an optionally substituted C₁-C₁₀ alkyl-OC(O)NH—, an optionally substituted (C₆₋₁₀ aryl)-C(O)O—, an optionally substituted (C₆₋₁₀ aryl)-C(O)NH—, an optionally substituted (C₆₋₁₀ aryl)-OC(O)NH—, an optionally substituted C₁-C₁₀ alkyl-OCH₂NH—, and an optionally substituted C₁-C₁₀ alkyl-OCH₂O—; and R¹² is selected from a group of H, NH₂, an optionally substituted C₁-C₁₀ alkylamino, an optionally substituted C₁-C₁₀ acylamino, an optionally substituted C₁-C₁₀ alkyl-OC(O)NH—, an optionally substituted (C₆₋₁₀ aryl)-C(O)NH—, an optionally substituted (C₆₋₁₀ aryl)-OC(O)NH—, and an optionally substituted C₁-C₁₀ alkyl-OCH₂NH—. In other embodiments, Base is

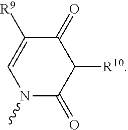

In other embodiments, R⁹ is methyl. In other embodiments, R¹⁰ is H.

Some embodiments relate to a compound selected from the group consisting of:

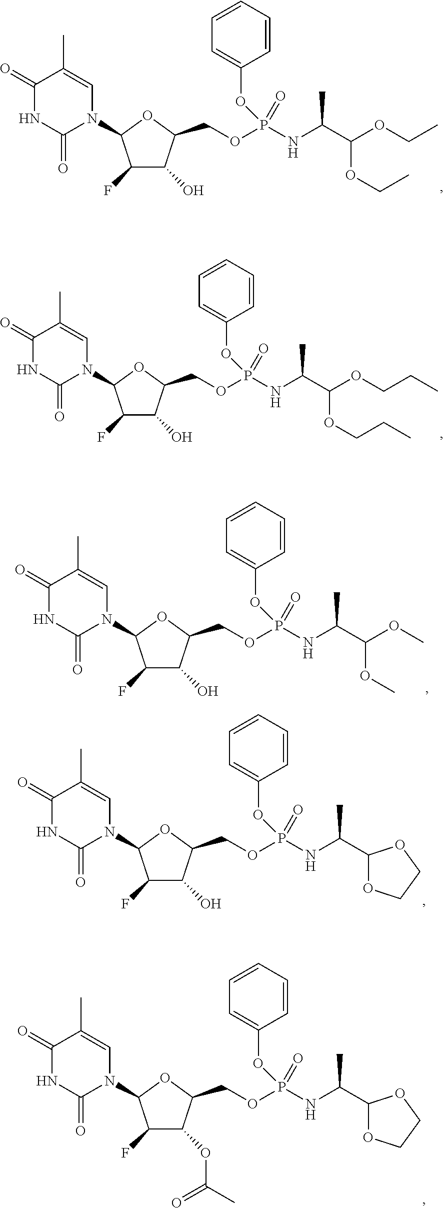 [SMILES: CCOC(OCC)[C@H](C)NP(=O)(OC[C@@H]1O[C@H](n2cc(C)c(=O)[nH]c2=O)[C@H](F)[C@H]1O)Oc1ccccc1 ; CCCOC(OCCC)[C@H](C)NP(=O)(OC[C@@H]1O[C@H](n2cc(C)c(=O)[nH]c2=O)[C@H](F)[C@H]1O)Oc1ccccc1 ; COC(OC)[C@H](C)NP(=O)(OC[C@@H]1O[C@H](n2cc(C)c(=O)[nH]c2=O)[C@H](F)[C@H]1O)Oc1ccccc1 ; Cc1cn([C@H]2O[C@@H](COP(=O)(N[C@@H](C)C3OCCO3)Oc3ccccc3)[C@H](O)[C@H]2F)c(=O)[nH]c1=O ; CC(=O)O[C@@H]1[C@@H](F)[C@@H](n2cc(C)c(=O)[nH]c2=O)O[C@H]1COP(=O)(N[C@@H](C)C1OCCO1)Oc1ccccc1]

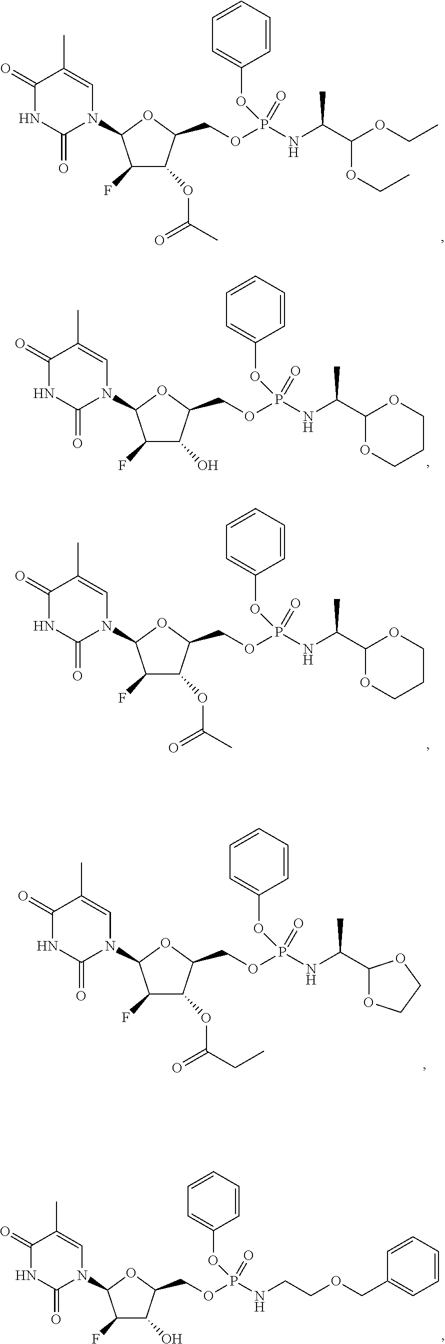 [SMILES: CCOC(OCC)[C@H](C)NP(=O)(OC[C@@H]1O[C@H](n2cc(C)c(=O)[nH]c2=O)[C@H](F)[C@H]1OC(C)=O)Oc1ccccc1 ; Cc1cn([C@H]2O[C@@H](COP(=O)(N[C@@H](C)C3OCCCO3)Oc3ccccc3)[C@H](O)[C@H]2F)c(=O)[nH]c1=O ; CC(=O)O[C@@H]1[C@@H](F)[C@@H](n2cc(C)c(=O)[nH]c2=O)O[C@H]1COP(=O)(N[C@@H](C)C1OCCCO1)Oc1ccccc1 ; CCC(=O)O[C@@H]1[C@@H](F)[C@@H](n2cc(C)c(=O)[nH]c2=O)O[C@H]1COP(=O)(N[C@@H](C)C1OCCO1)Oc1ccccc1 ; Cc1cn([C@H]2O[C@@H](COP(=O)(NCCOCc3ccccc3)Oc3ccccc3)[C@H](O)[C@H]2F)c(=O)[nH]c1=O]

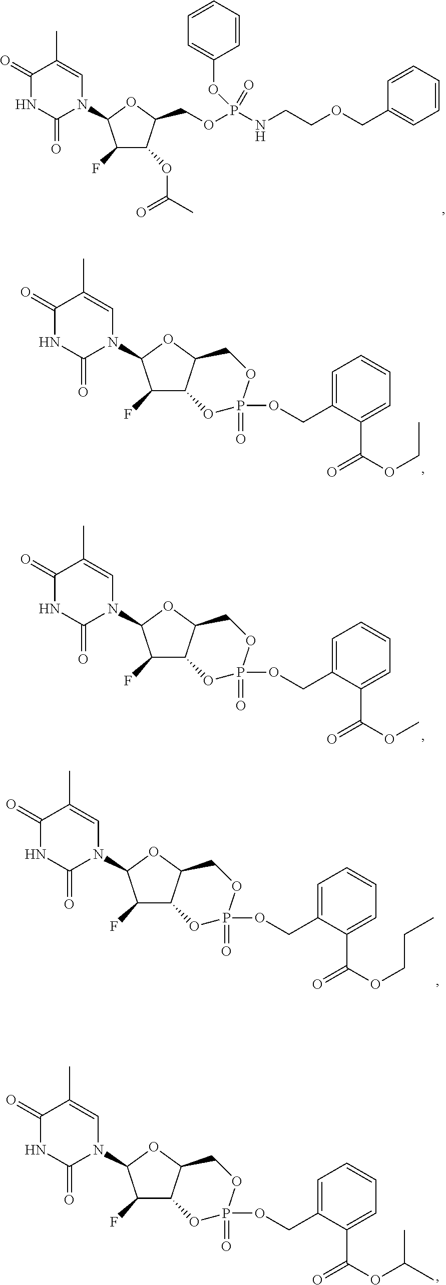

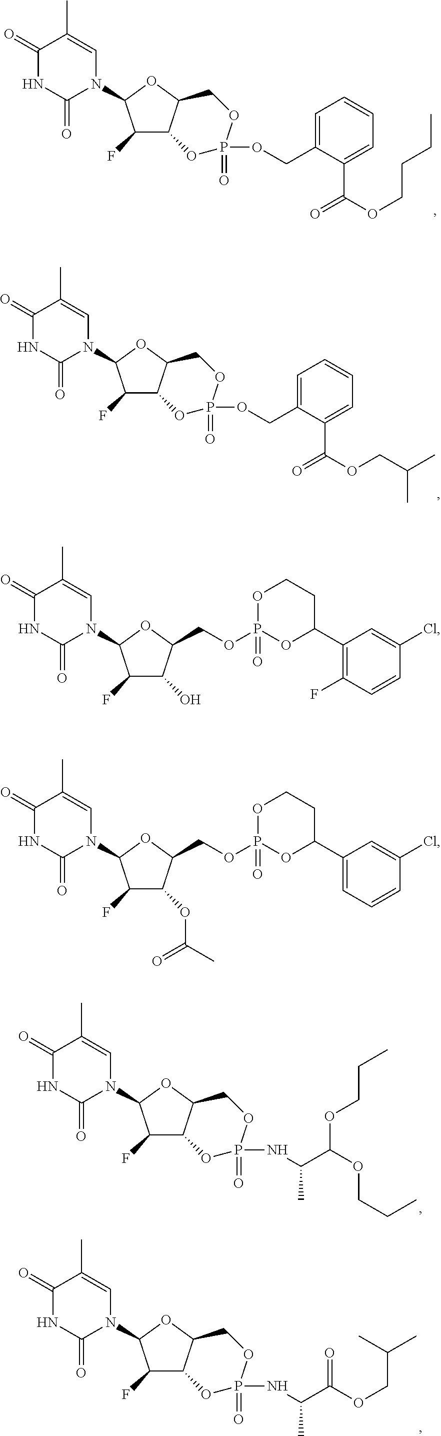

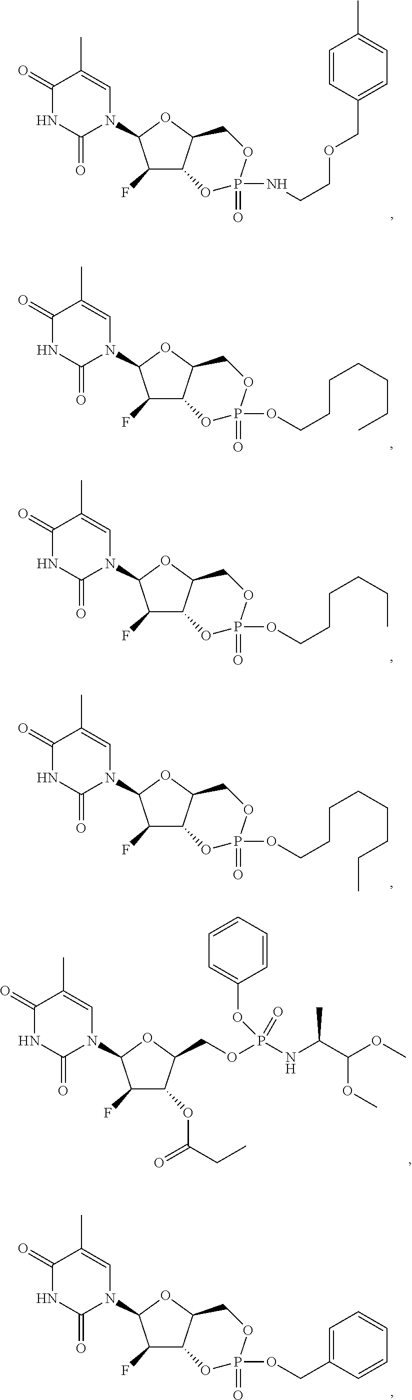

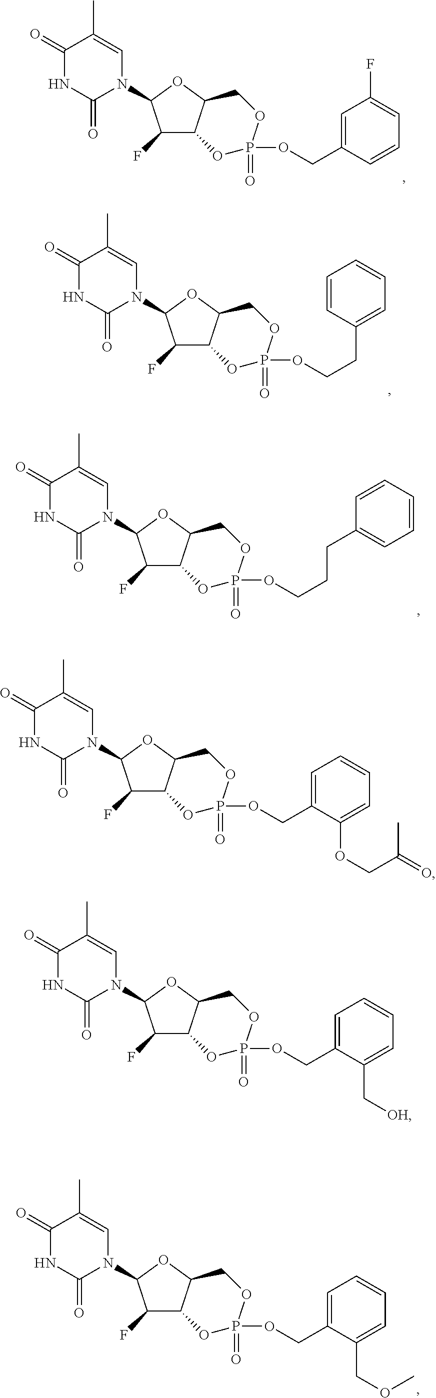

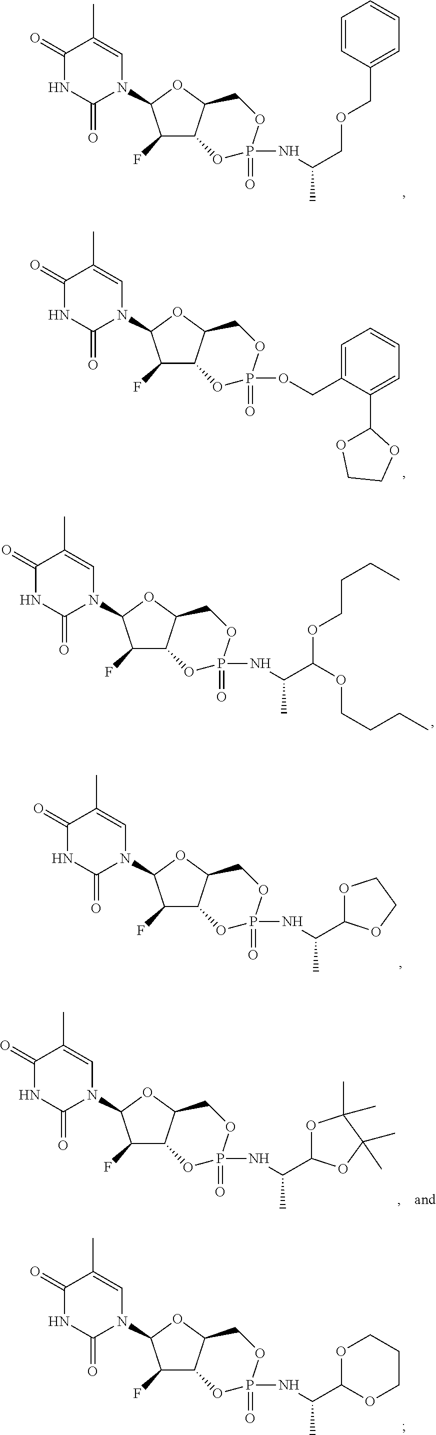

or a stereoisomer or a pharmaceutically acceptable salt thereof.

Further embodiments relate to pharmaceutical composition comprising a compound as described herein and a pharmaceutically acceptable excipient.

Some embodiments further comprise one or more anti-cancer agents.

Some embodiments relate to a method of treating a disease, disorder or condition comprising administering an effective amount of a compound as described herein to a subject in need thereof. In other embodiments, the disease, disorder, or condition is a disease, disorder, or condition of the liver. In other embodiments, the disease, disorder or condition is a metabolic, cardiovascular, or hormonal disease in which the liver is involved in the production and/or the homeostasis control of the biochemical end products of the disease, disorder, or condition. In other embodiments, the disease, disorder or condition is selected from the group consisting of hepatitis, cancer, liver fibrosis, fatty liver, malaria, viral infection, parasitic infection, diabetes, hyperlipidemia, atherosclerosis, obesity, dyslipidemia, hyperglycemia, and a hormonal condition. In other embodiments, the disease, disorder or condition is a viral infection, cancer, or other disease in which the prodrug compounds enhance the distribution of an active drug to the target tissue or cell.

In some embodiments, the subject is a mammal. In other embodiments, the subject is a human.

Some embodiments relate to a method as described herein, further comprising administering an effective amount of at least one additional therapeutic agent to the subject in need thereof. In other embodiments, the additional therapeutic agent is one or more of sorafenib, regorafenib, an immune-oncology agent such as a PD-1 or PD-L1 checkpoint inhibitor.

Some embodiments relate to a compound as described herein for use in treating a disease or condition in the liver or a disease or condition in which the physiological or pathogenic pathways involve the liver.

Some embodiments relate to the use of a compound as described herein in the preparation of a medicament for treating a disease or condition in the liver or a disease or condition in which the physiological or pathogenic pathways involve the liver.

In some embodiments, Base is a natural or modified nuclear base such as adenine, guanine, thymine, uracil, cytosine, and their analogs.

In some embodiments, the phosphoramidate and cyclophosphate compounds of Formula I, II, III, IV, V, VI, and VII are substrates of liver enzymes such as cytochrome p450 isozymes CYP3As (a family of monooxygenase), dehydrogenases, esterases, and/or amidases.

CYP3A4 is expressed in the liver in a level much higher than other tissues (DeWaziers et al. J Pharm Exp Ther 253.387 (1990)). Phosphoramidate and cyclophosphate compounds of Formula I, IV, V. and VI are predominantly activated via CYP3A4 in the liver. Cyclophosphate compounds of Formula II and III are predominantly activated via the esterase in the liver. In some embodiments, the compounds of Formula I, II, III, IV, V, VI, VII have high efficiency in liver-targeting via selective delivery of biologically active agents to the liver. In some embodiments, the phosphoramidate and cyclophosphate compounds are used to increase the therapeutic index of a drug, since the compounds of Formula I, II, III, IV, V. VI, and VII may not be active or may be less active outside the liver.

In some embodiments, due to the liver-targeting nature of the phosphoramidate and cyclophosphate compounds of Formula I, II, W, IV, V, VI, and VII, the compounds are used to treat diseases that benefit from enhanced drug distribution to the liver and like tissues and cells, including but not limited to diseases in the liver, such as hepatitis, liver cancer, liver fibrosis, fatty liver, malaria, other viral and parasitic infections, and metabolic, cardiovascular, and/or hormonal diseases where the liver is involved in the production and/or the homeostasis control of biochemical end products, e.g. glucose (e.g. diabetes); cholesterol, fatty acids, bile acids, triglycerides (e.g. hyperlipidemia, atherosclerosis and obesity), lipoproteins, apolipoproteins, and sex hormone-binding globulin (SHBG).

In some embodiments, the disclosed compounds are used to improve pharmacokinetic properties such as prolonging half-life or enhancing absorption of a drug. In addition, the disclosed methodology can be used to achieve sustained delivery of an active therapeutic agent. Due to the pharmacokinetic property enhancement of the phosphoramidate and cyclophosphate compounds of Formula I, II, III, IV, V, VI, VII, the compounds are used to treat diseases that benefit from enhanced drug properties, including but not limited to diseases such as HIV, viral infection, and cancer. In some embodiments, a method of making these compounds is described. In some embodiments, the compounds are also useful in the delivery of diagnostic imaging agents to the liver or other tissues.

Certain compounds of Formula I, II, III, IV, V, VI, and VII have an asymmetric center at the phosphorus atom where the stereochemistry may be unspecified, and the diastereomeric mixtures of these compounds are included, as well as the individual stereoisomers when referring to a compound of Formula I, II, III, IV, V. VI, and VII generally.

Some embodiments of the compounds, compositions and methods provided herein include a pharmaceutical composition comprising a compound provided herein and a pharmaceutically acceptable carrier.

Some embodiments also include administering an effective amount of a second or multiple therapeutic agents in combination with a compound provided herein to the subject in need thereof.

In some embodiments, the subject is mammalian.

In some embodiments, the subject is human.

Some embodiments of the compounds, compositions and methods provided herein include a method of testing a compound in a cell comprising contacting the cell with the disclosed compounds.

Some embodiments of the compounds, compositions and methods provided herein include use of a compound provided herein in the treatment of a disease in the liver.

Some embodiments include the use of a compound provided herein in combination with additional therapeutic agent(s) for the treatment of a disease in the liver.

Some embodiments of the compounds, compositions and methods provided herein include use of a compound provided herein in the treatment of a non-liver disease such as HIV and other viral infections as well as cancer.

Some embodiments include the use of a compound provided herein in combination with additional therapeutic agent(s) for the treatment of a non-liver disease such as HIV and other viral infections as well as cancer.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

### Definitions

In accordance with the present disclosure and as used herein, the following terms are defined with the following meanings, unless explicitly stated otherwise. It is to be understood that both the foregoing general description and the following detailed description are exemplary and explanatory only and are not restrictive of the subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included” is not limiting.

As used herein, ranges and amounts can be expressed as “about” a particular value or range. “About” also includes the exact amount. Hence “about 10%” means “about 10%” and also “10%.”

As used herein, “optional” or “optionally” means that the subsequently described event or circumstance does or does not occur, and that the description includes instances where the event or circumstance occurs and instances where it does not. For example, an optionally substituted group means that the group is unsubstituted or is substituted.

As used herein, the singular forms “a,” “an” and “the” include plural referents unless the context clearly dictates otherwise. Thus, for example, reference to a composition comprising “a therapeutic agent” includes compositions with one or a plurality of therapeutic agents.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₂-C₆ alkenyl, C₂-C₆ alkynyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocycyl (optionally substituted with halo, C₁-C₆ alkyl, C₁-C₆ alkoxy, C₁-C₆ haloalkyl, and C₁-C₆ haloalkoxy), 3-10 membered heterocycyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo. C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo. C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₁-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

As used herein, “acyl” refers to —C(S)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(C)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “S-sulfonamide” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-sulfonamide” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein each optionally substituted with one or more substituents selected from the group consisting of —OH, C₁₋₆ alkyl, C₃₋₇ carbocyclyl, C₆₋₁₀ aryl, 5-10 membered heteroaryl, 3-10 membered heterocycyl, C₁₋₆ alkyl optionally substituted with C₁₋₆ alkoxy or —OH and C₁₋₆ alkoxy optionally substituted with C₁₋₆ alkoxy or —OH.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein each optionally substituted with one or more substituents selected from the group consisting of —OH, C₁₋₆ alkyl, C₃₋₇ carbocyclyl, C₆₋₁₀ aryl, 5-10 membered heteroaryl, 3-10 membered heterocycyl. C₁₋₆ alkyl optionally substituted with C₁-6 alkoxy or —OH and C₁₋₆ alkoxy optionally substituted with C₁₋₆ alkoxy or —OH.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

The term “acyloxy” refers to —OC(O)R where R is alkyl.

The term “alkoxy” or “alkyloxy” refers to OR where R is alkyl, or heteroalkyl, all optionally substituted.

The term “carboxyl” refers to a C(O)OH.

The term “oxo” refers to an ═O group.

The term “halogen” or “halo” refers to F (fluoro), Cl (chloro), Br (bromo) and I (iodo).

The term “haloalkyl” refer to alkyl groups containing at least one halogen, in a further aspect are 1 to 3 haloatoms. Suitable haloatoms include F, Cl, and Br.

The term “haloacyl” refer to —C(O)-haloalkyl groups.

The term “alkenyl” refers to unsaturated groups which have 2 to 12 atoms and contain at least one carbon carbon double bond and includes straight chain, branched chain and cyclic groups. Alkenyl groups may be optionally substituted. Suitable alkenyl groups include allyl.

The term “alkynyl” refers to unsaturated groups which have 2 to 12 atoms and contain at least one carbon carbon triple bond and includes straight chain, branched chain and cyclic groups. Alkynyl groups may be optionally substituted. Suitable alkynyl groups include ethynyl.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Heteroaryl groups may be optionally substituted. Examples of heteroaryl groups include, but are not limited to, aromatic C3-8 heterocyclic groups comprising one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In some embodiments, heteroaryl groups are optionally substituted with one or more substituents, independently selected from halo, hydroxy, amino, cyano, nitro, alkylamido, acyl, C₁₋₆-alkoxy, C₁₋₆-alkyl, C₁₋₆ hydroxyalkyl, C1-6-aminoalkyl, C1-6-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

As used herein, “cycloalkyl” means a fully saturated carbocyclyl ring or ring system. Examples include cyclopropyl, cyclobutyl, cyclopentyl, and cyclohexyl. The cycloalkyl group may have 3 to 10 carbon atoms (whenever it appears herein, a numerical range such as “3 to 10” refers to each integer in the given range. The cycloalkyl group may be designated as “C₃-C₈ cycloalkyl” or similar designations. By way of example only, “C₃-C₈ cycloalkyl” indicates that there are three to eight carbon atoms in the carbocyclyl ring or ring system.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O (oxygen), N (nitrogen) or S (sulfur), and in preferred five membered monocyclic heterocyclyls, the heteroatom(s) are selected from one or two heteroatoms selected from O (oxygen), N (nitrogen) or S (sulfur). Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atoms to which they are attached,” it is meant that the collective unit of the atoms and the two R groups are the recited ring. The ring is not otherwise limited by the definition of each R group when taken individually. For example, when the following substructure is present:

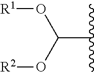

and R¹ and R² are defined as selected from the group consisting of alkyl and aryl, or R¹ and R² together with the oxygen to which they are each attached and intervening atom(s) form a heterocyclyl, it is meant that R¹ and R² can be selected from alkyl or aryl, or alternatively, the substructure has structure:

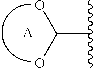

where ring A is a heterocyclic ring containing the depicted oxygens.

Similarly, when two “adjacent” R groups are said to form a ring “together with the atom to which they are attached,” it is meant that the collective unit of the atoms, intervening bonds, and the two R groups are the recited ring. For example, when the following substructure is present:

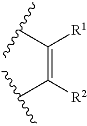

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

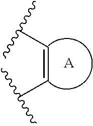

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

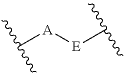

includes the substituent being oriented such that the A is attached at the leftmost attachment point of the molecule as well as the case in which A is attached at the rightmost attachment point of the molecule.

The phrase “therapeutically effective amount” means an amount of a compound or a combination of compounds that partially or fully ameliorates, attenuates or eliminates one or more of the symptoms of a particular disease or condition or prevents, modifies, or delays the onset of one or more of the symptoms of a particular disease or condition. Such amount can be administered as a single dosage or can be administered according to a regimen, whereby it is effective. Repeated administration may be needed to achieve a desired result (e.g., treatment of the disease and/or condition).

The term “pharmaceutically acceptable salt” includes salts of compounds of Formula I, II, III and IV derived from the combination of a compound of the present embodiments and an organic or inorganic acid or base. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, adipic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzenesulfonic acid, methanesulfonic acid, ethanesulfonic acid, p-toluenesulfonic acid, (+)-7,7-dimethyl-2-oxobicyclo[2.2.1]heptane-1-methanesulfonic acid, 1,2-ethanedisulfonic acid, dodecyl sulfonic acid, salicylic acid, glucoheptonic acid, gluconic acid, glucuronic acid, hippuric acid, hydrochloride hemiethanolic acid, 2-hydroxyethanesulfonic acid, lactic acid, lactobionic acid, methylbromide acid, methyl sulfuric acid, 2-naphthalenesulfonic acid, oleic acid, 4,4′-methylenebis-[3-hydroxy-2-naphthalenecarboxylic acid], polygalacturonic acid, stearic acid, sulfosalicylic acid, tannic acid, terphthalic acid and the like. Inorganic bases from which salts can be derived include, for example, bases that contain sodium, potassium, lithium, ammonium, calcium, magnesium, iron, zinc, copper, manganese, aluminum, and the like, particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

Where the number of any given substituent is not specified (e.g., “haloalkyl”), there may be one or more substituents present. For example, “haloalkyl” can include one or more of the same or different halogens. For example, “haloalkyl” includes each of the substituents CF₃, CHF₂ and CH₂F.

The term “patient” refers to an animal being treated including a mammal, such as a dog, a cat, a cow, a horse, a sheep, and a human. In some embodiments, the patient is a mammal, either male or female. In some embodiments, the patient is a male or female human.

The term “stereoisomer” refers to the relative or absolute spatial relationship of the R group(s) attached to the stereogenic centers either carbon or phosphorus atoms, and refers to individual or any combination of the individual isomers such as a racemic mixture and a diastereomeric mixture. When a compound has two stereogenic centers, there are 4 potential stereoisomers.

The term “liver” refers to the liver organ.

The term “liver specificity” refers to the ratio:
[drug or a drug metabolite in liver tissue]/[drug or a drug metabolite in blood or another tissue]

as measured in animals treated with the drug or a prodrug. The ratio can be determined by measuring tissue levels at a specific time or may represent an AUC (area under a curve) based on values measured at three or more time points.

The term “increased or enhanced liver specificity” refers to an increase in liver specificity ratio in animals treated with the prodrug relative to animals treated with the parent drug.

The term “enhanced oral bioavailability” refers to an increase of at least about 50% of the absorption of the dose of the reference drug. In an additional aspect, the increase in oral bioavailability of the compound (compared to the reference drug) is at least about 100%, or a doubling of the absorption. Measurement of oral bioavailability usually refers to measurements of the prodrug, drug, or drug metabolite in blood, plasma, tissues, or urine following oral administration compared to measurements following parenteral administration.

The term “therapeutic index” refers to the ratio of the dose of a drug or prodrug that produces a therapeutically beneficial response relative to the dose that produces an undesired response such as death, an elevation of markers that are indicative of toxicity, and/or pharmacological side effects.

The term “sustained delivery” refers to an increase in the period in which there is a prolongation of therapeutically-effective drug levels due to the presence of the prodrug.

The terms “treating” or “treatment” of a disease includes inhibiting the disease (slowing or arresting or partially arresting its development), preventing the disease, providing relief from the symptoms or side effects of the disease (including palliative treatment), and/or relieving the disease (causing regression of the disease).

The terms “molecular pathway” refers to a series of molecular events in tissues such as a receptor modulating sequence, an enzyme modulating sequence, or a biosynthesis sequence that is involved in physiological or pathophysiological functions of a living animal.

### Administration and Pharmaceutical Compositions

The disclosed compounds may be used alone or in combination with other treatments. These compounds, when used in combination with other agents, may be administered as a daily dose or an appropriate fraction of the daily dose (e.g., bid). The compounds may be administered after a course of treatment by another agent, during a course of therapy with another agent, administered as part of a therapeutic regimen, or may be administered prior to therapy with another agent in a treatment program.

Examples of pharmaceutically acceptable salts include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Compositions containing the active ingredient may be in any form suitable for the intended method of administration. In some embodiments, the compounds of a method and/or composition described herein can be provided via oral administration, rectal administration, transmucosal administration, intestinal administration, enteral administration, topical administration, transdermal administration, intrathecal administration, intraventricular administration, intraperitoneal administration, intranasal administration, intraocular administration and/or parenteral administration.

When the compounds are administered via oral administration, for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient can be mixed with an inert solid diluent, for example calcium phosphate or kaolin, or as soft gelatin capsules wherein the active ingredient can be mixed with water or an oil medium, such as peanut oil, liquid paraffin or olive oil.

Formulations suitable for parenteral administration include aqueous and non-aqueous isotonic sterile injection solutions which may contain, for example, antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

In some embodiments unit dosage formulations contain a daily dose or unit, daily sub-dose, or an appropriate fraction thereof, of a drug. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

The actual dose of the compounds described herein depends on the specific compound, and on the condition to be treated; the selection of the appropriate dose is well within the knowledge of the skilled artisan. In some embodiments, a daily dose may be from about 0.1 mg/kg to about 100 mg/kg or more of body weight, from about 0.25 mg/kg or less to about 50 mg/kg, from about 0.5 mg/kg or less to about 25 mg/kg, from about 1.0 mg/kg to about 10 mg/kg of body weight. Thus, for administration to a 70 kg person, the dosage range would be from about 7 mg per day to about 7000 mg per day, from about 35 mg per day or less to about 2000 mg per day or more, from about 70 mg per day to about 1000 mg per day.

### Methods of Treatment

Some embodiments of the present invention include methods of treating a disease, disorder or condition is selected from the group consisting of hepatitis, liver cancer, liver fibrosis, fatty liver, malaria, viral infection, parasitic infection, diabetes, hyperlipidemia, atherosclerosis, obesity, dyslipidemia, hyperglycemia, a hormonal condition, HIV, and various types of cancer with the compounds, and compositions comprising compounds described herein. Some methods include administering a compound, composition, pharmaceutical composition described herein to a subject in need thereof. In some embodiments, a subject can be an animal, e.g., a mammal, a human. In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament or additional therapeutic agent(s). By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment, the agents are administered through the same route, such as orally. In another embodiment, the agents are administered through different routes, such as one being administered orally and another being administered i.v.

Examples of additional medicaments include a therapeutic agent(s) selected from the group consisting of thymosin alpha-1, interferon-A, an inhibitor of HCV protease, an inhibitor of HCV NSSA replication complex, an inhibitor of HCV NSSB polymerase, an inhibitor of HCV helicase, a cyclophilin inhibitor, an inhibitor of inosine monophosphate dehydrogenase, ribavirin, interferon-α, and pegylated interferon-α. In some embodiments, additional medicaments include one or more of cobicistat, emtricitabine and elvitegravir. In some embodiments, additional medicaments include two or more of cobicistat, emtricitabine and elvitegravir. In some embodiments, additional medicaments include cobicistat, emtricitabine and elvitegravir. In some embodiments, additional medicaments include one or more of ribavirin, peginterferon-alfa, simeprevir, ledipasvir and daclatasvir. In some embodiments, the additional therapeutic agent may be one or more of cobicistat, emtricitabine and elvitegravir. In some embodiments, the additional therapeutic agent may be two or more of cobicistat, emtricitabine and elvitegravir. In some embodiments, the additional therapeutic agent may be cobicistat, emtricitabine and elvitegravir. In some embodiments, the additional therapeutic agent may be one or more of ribavirin, peginterferon-alfa, simeprevir, ledipasvir and daclatasvir. In some embodiments, the additional therapeutic agent for HBV treatment may be one or more of a HBV entry inhibitor, a HBV cccDNA inhibitor, a HBV capsid inhibitor, an interferon, HBV assembly inhibitor. In some embodiments, the additional therapeutic agent for HCC treatment may be one or more of sorafenib, regorafenib, an immune-oncology agent such as a PD-1 or PD-L1 checkpoint inhibitor.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Synthesis of Compounds

The following procedures for the preparation of the new compounds illustrate the general procedures used to prepare the phosphoramidate and cyclophosphate compounds. A protecting group can be introduced at different stages of synthesis of a drug. In some embodiments, they are introduced at a later stage, because of the general sensitivity of these groups to various reaction conditions. Optically pure compounds containing a single isomer at the phosphorus center can be made, for example, by separation of the diastereomers by a combination of column chromatography and/or crystallization, or by enantioselective synthesis of chiral activated phosph(on)ate intermediates.

Scheme II describes general strategies of synthesis of the compounds of Formula I. The compound of structure 1 is condensed with an unnatural nucleoside of structure 2 in the presence of a base to give a product of structure 3. Amino compound of structure 4 is prepared from the corresponding aldehyde or ether by the standard procedure in the literature. Reaction of the compounds of structures 3 and 4 in the presence of a base affords the final product of structure 5. Alternatively, the aryloxy phosphate chloride of structure 1 is couple with amino compound of structure 4 first and then with the nucleoside of structure 2 to yield the final product of structure 5.

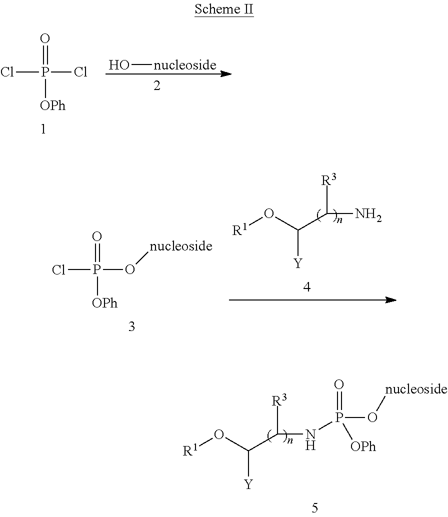

Scheme III describes general synthesis of the compounds of Formula II, V. and VI. Nucleoside of structure 6 reacts with the phosphanediamine (7) in the presence of 4,5-dicyanoimidazole to give the cyclic product of structure 8 and the crude reaction mixture is then treated with an oxidation agent such as tert-butyl hydroperoxide to afford the final product of structure 9 of Formula II, V. and VI.

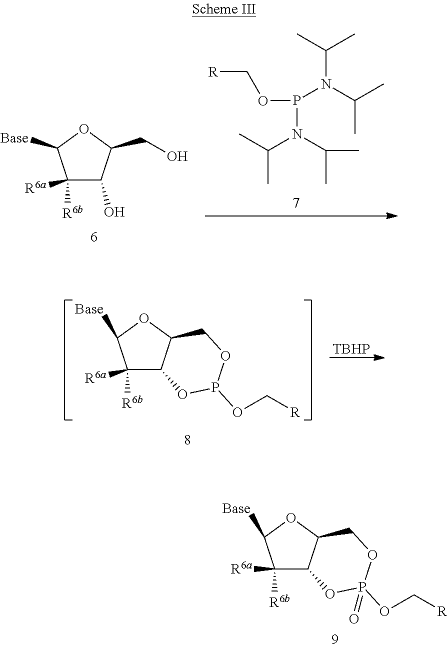

R=

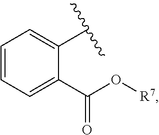

alkyl, or phenyl derivatives

Scheme IV describes general synthesis of the compounds of Formula III and IV. The compound of structure 1 where the Ph group is substituted with electron-withdrawing group(s) is condensed with an unnatural nucleoside of structure 2 in the presence of a base to give a product of structure 3. Amino compounds of structure 4 or 4a are prepared from the corresponding aldehyde or ether by the standard procedure in the literature. Reaction of the compounds of structures 3 and 4 (or 4a) in the presence of a base affords the intermediates of structure 5 (or 5a). Treatment of intermediate compounds of structure 5 (or 5a) with an organic base such as t-BuOK in a polar solvent provide final products of structure 10 (or 11).

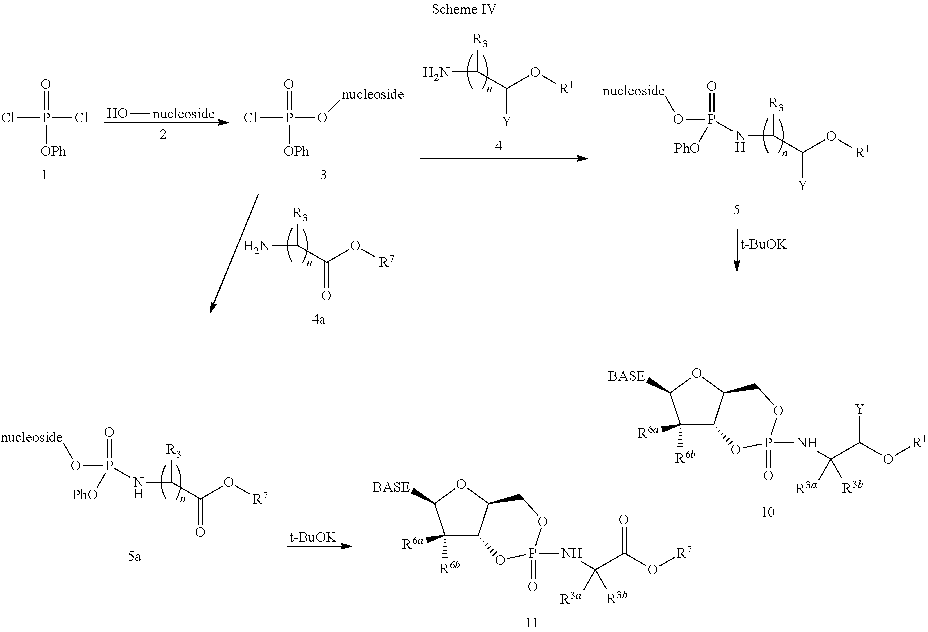

### EXAMPLES

Compounds of Formula I and II are prepared as outlined below.

### Example 1

### ((2S,3S,4R,5S)-4-Fluoro-3-hydroxy-5-(5-methyl-2,4-dioxo-3,4-dihydropyrimidin-1(2H)-yl)tetrahydrofuran-2-yl)methyl phenyl ((S)-1,1-diethoxypropan-2-yl)phosphoramidate (Compound 101)

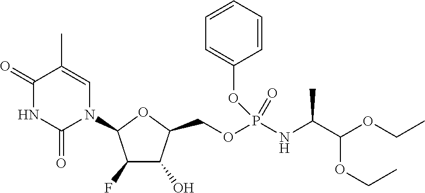 [SMILES: CCOC(OCC)[C@H](C)NP(=O)(OC[C@@H]1O[C@H](n2cc(C)c(=O)[nH]c2=O)[C@H](F)[C@H]1O)Oc1ccccc1]

Compound 101 was prepared according to Scheme II from phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione as follows.

### (S)—N-Cbz-2-aminopropanal

(S)—N-Cbz-2-aminopropanol (15 g, 72 mmol) was refluxed with 2-iodoxybenzoic acid (IBX) (41 g, 144 mmol) in ACN (350 mL) for 2-4 hours. After completion of the reaction (monitored by TLC), the IBX was filtered through celite. The filtrate was concentrated to dryness under vacuum to give the product (11.4 g, 94%).

### (S)-1,1-Diethoxypropan-2-amine

A suspension mixture of the above aldehyde (5.0 g, 24 mmol), PPTS (0.6 g, 2.4 mmol), and triethyl orthoformate (7.2 g, 48 mmol) in ethanol (50 mL) was stirred at 90° C. for 16 hours. The reaction mixture was washed sequentially with aqueous HCl (1M), NaHCO₃ (saturated), and brine, dried over Na₂SO₄, and concentrated. Silica gel chromatography (PE:EA=4:1) of the crude gave the N-Cbz protected product in 50% yield (3.0 g). A suspension mixture of the N-Cbz-amine (2 g) and activated Pd/C (20% by weight) in ethyl acetate (30 mL) was stirred overnight at room temperature in the presence of hydrogen gas (H2 balloon). The Pd/C was filtered through celite and the filtrate was concentrated to dryness under vacuum to give 1.0 g g of the product (56% yield).

### Phenyl ((S)-1,1-diethoxypropan-2-yl)phosphoramidochloridate

To a solution of phenyl phosphorodichloridate (0.4 g, 2.0 mmol) in dichloromethane at −60° C. was added the above compound (0.3 g, 2.0 mmol) in the presence of excess triethylamine and the resulting mixture was stirred for 2 hours. Standard work-up procedure afforded the crude product in ˜60% yield (0.6 g, 1.8 mmol). Compound 101:

To a solution of the crude phenyl phosphoramidochloridate in THF (0.5 mL) was added a solution of 1-((2S,3R,4S,5S)-3-fluoro-4-hydroxy-5-(hydroxymethyl)tetrahydrofuran-2-yl)-5-methylpyrimidine-2,4(1H,3H)-dione (0.1 g, 0.4 mmol) and t-BuMgCl THF solution (1 mmol) in THF (0.5 mL) at −78° C. under nitrogen. The reaction mixture was slowly allowed to warm to room temperature and stirred for one hour. Standard work-up followed by Pre-HPLC to give compound 101 as a mixture of two diastereomers (20 mg).

[M−H]⁺ calculated for C₂₃H₃₃FN₃O₉P: 544.18; Found: 544.2. ¹H NMR (400 MHz, acetone-d₆) 10.13 (bs, 1H), 7.70-7.15 (m, 6H), 6.20-6.26 (m, 1H), 5.52-5.10 (m, 2H), 4.60-4.15 (m, 4H), 3.95-3.45 (m, 7H), 1.82 (s, 3H), and 1.20-1.00 (m, 9H).

### Example 2

### ((2S,3 S,4R,5S)-4-Fluoro-3-hydroxy-5-(5-methyl-2,4-dioxo-3,4-dihydropyrimidin-1(2H)-yl)tetrahydrofuran-2-yl)methyl phenyl ((S)-1,1-dipropoxypropan-2-yl)phosphoramidate (Compound 102)

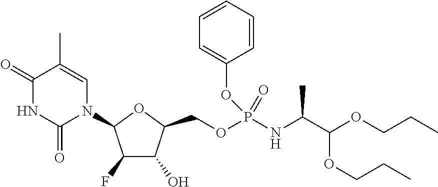 [SMILES: CCCOC(OCCC)[C@H](C)NP(=O)(OC[C@@H]1O[C@H](n2cc(C)c(=O)[nH]c2=O)[C@H](F)[C@H]1O)Oc1ccccc1]

Compound 102 can be prepared according to Scheme II from phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₅H₃₇FN₃O₉P: 572.22.

### Example 3

### ((2S,3S,4R,5S)-4-Fluoro-3-hydroxy-5-(5-methyl-2,4-dioxo-3,4-dihydropyrimidin-1(2H)-yl)tetrahydrofuran-2-yl)methyl phenyl ((S)-1,1-dimethoxypropan-2-yl)phosphoramidate (Compound 103)

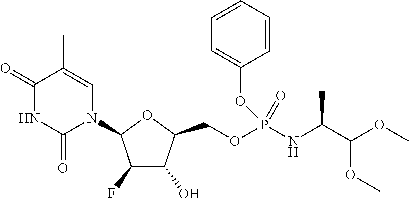 [SMILES: COC(OC)[C@H](C)NP(=O)(OC[C@@H]1O[C@H](n2cc(C)c(=O)[nH]c2=O)[C@H](F)[C@H]1O)Oc1ccccc1]

Compound 103 can be prepared according to Scheme II from phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₁H₂₉FN₃O₉P: 516.15.

### Example 4

### ((2S,3S,4R,5S)-4-Fluoro-3-hydroxy-5-(5-methyl-2,4-dioxo-3,4-dihydropyrimidin-1(2H)-yl)tetrahydrofuran-2-yl)methyl phenyl ((S)-1-(1,3-dioxolan-2-yl)ethyl)phosphoramidate (Compound 104)

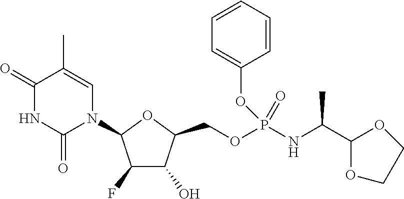 [SMILES: Cc1cn([C@H]2O[C@@H](COP(=O)(N[C@@H](C)C3OCCO3)Oc3ccccc3)[C@H](O)[C@H]2F)c(=O)[nH]c1=O]

Compound 104 was prepared according to Scheme II from phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₂₁H₂₇FN₃O₉P: 514.14, found: 514.2.

### Example 5

### (2S,3S,4R,5S)-2-((((((S)-1-(1,3-Dioxolan-2-yl)ethyl)amino)(phenoxy)phosphoryl)-oxy)methyl)-4-fluoro-5-(5-methyl-2,4-dioxo-3,4-dihydropyrimidin-1(2H)-yl)tetrahydrofuran-3-yl acetate (Compound 105)

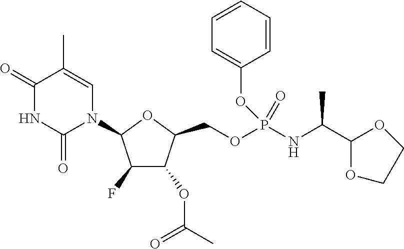 [SMILES: CC(=O)O[C@@H]1[C@@H](F)[C@@H](n2cc(C)c(=O)[nH]c2=O)O[C@H]1COP(=O)(N[C@@H](C)C1OCCO1)Oc1ccccc1]

Compound 105 was prepared by acylation of Compound 104 as a mixture of two diastereomers. [M−H]⁺ calculated for C₂₃H₂₉FN₃O₁₀P: 556.15; found: 556.2.

### Example 6

### (2S,3S,4R,5S)-2-((((((S)-1,1-Diethoxypropan-2-yl)amino)(phenoxy)phosphoryl)oxy)methyl)-4-fluoro-5-(5-methyl-2,4-dioxo-3,4-dihydropyrimidin-1(2H)-yl)tetrahydrofuran-3-yl Acetate (Compound 106)

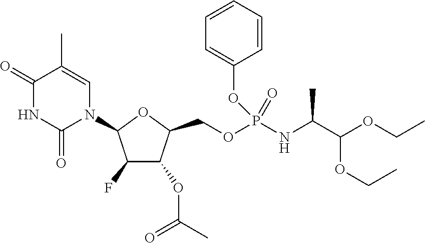 [SMILES: CCOC(OCC)[C@H](C)NP(=O)(OC[C@@H]1O[C@H](n2cc(C)c(=O)[nH]c2=O)[C@H](F)[C@H]1OC(C)=O)Oc1ccccc1]

Compound 106 was prepared by acylation of Compound 101. [M−H]⁺ calculated for C₂₅H₃₅FN₃O₁₀P: 586.19; Found: 586.2.

### Example 7

### ((2S,3 S,4R,5S)-4-Fluoro-3-hydroxy-5-(5-methyl-2,4-dioxo-3,4-dihydropyrimidin-1(2H)-yl)tetrahydrofuran-2-yl)methyl phenyl ((S)-1-(1,3-dioxan-2-yl)ethyl)phosphoramidate (Compound 107)

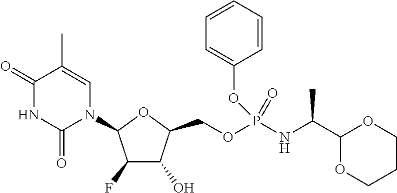 [SMILES: Cc1cn([C@H]2O[C@@H](COP(=O)(N[C@@H](C)C3OCCCO3)Oc3ccccc3)[C@H](O)[C@H]2F)c(=O)[nH]c1=O]

Compound 107 can be prepared according to Scheme II from phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₂H₂₉FN₃O₉P: 528.45.

### Example 8

### (2S,3S,4R,5S)-2-((((((S)-1-(1,3-Dioxan-2-yl)ethyl)amino)(phenoxy)phosphoryl)oxy)-methyl)-4-fluoro-5-(5-methyl-2,4-dioxo-3,4-dihydropyrimidin-1(2H)-yl)tetrahydrofuran-3-yl acetate (Compound 108)

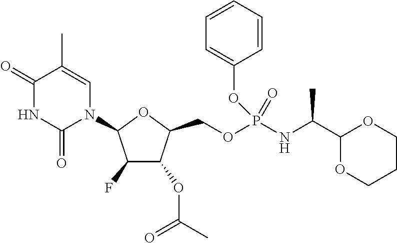 [SMILES: CC(=O)O[C@@H]1[C@@H](F)[C@@H](n2cc(C)c(=O)[nH]c2=O)O[C@H]1COP(=O)(N[C@@H](C)C1OCCCO1)Oc1ccccc1]

Compound 108 can be prepared by acylation of Compound 107. [M−H]⁺ calculated for C₂₄H₃₁FN₃O₁₀P: 570.16.

### Example 9

### (2S,3S,4R,5S)-2-((((((S)-1-(1,3-Dioxolan-2-yl)ethyl)amino)(phenoxy)phosphoryl)-oxy)methyl)-4-fluoro-5-(5-methyl-2,4-dioxo-3,4-dihydropyrimidin-1(2H)-yl)tetrahydrofuran-3-yl propionate (Compound 109)

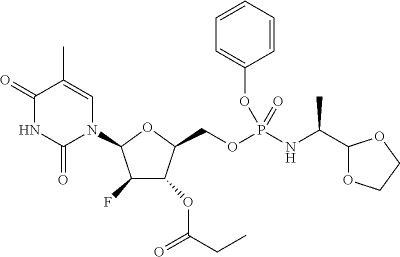 [SMILES: CCC(=O)O[C@@H]1[C@@H](F)[C@@H](n2cc(C)c(=O)[nH]c2=O)O[C@H]1COP(=O)(N[C@@H](C)C1OCCO1)Oc1ccccc1]

Compound 109 can be prepared by acylation of Compound 104. [M−H]⁺ calculated for C₂₄H₃₁FN₃O₁₀P: 570.16.

### Example 10

### ((2S,3S,4R,5S)-4-Fluoro-3-hydroxy-5-(5-methyl-2,4-dioxo-3,4-dihydropyrimidin-1(2H)-yl)tetrahydrofuran-2-yl)methyl phenyl (2-(benzyloxy)ethyl)phosphoramidate (Compound 110)

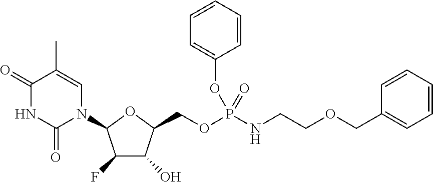 [SMILES: Cc1cn([C@H]2O[C@@H](COP(=O)(NCCOCc3ccccc3)Oc3ccccc3)[C@H](O)[C@H]2F)c(=O)[nH]c1=O]

Compound 110 was prepared according to Scheme II from phenyl phosphorodichloridate, 2-benzyloxyethylamine, and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₂₅H₂₉FN₃O₈P: 548.16; Found: 548.2.

### Example 11

### (2S,3S,4R,5S)-2-(((((2-(Benzyloxy)ethyl)amino)(phenoxy)phosphoryl)oxy)methyl)-4-fluoro-5-(5-methyl-2,4-dioxo-3,4-dihydropyrimidin-1(2H)-yl)tetrahydrofuran-3-yl acetate (Compound 111)

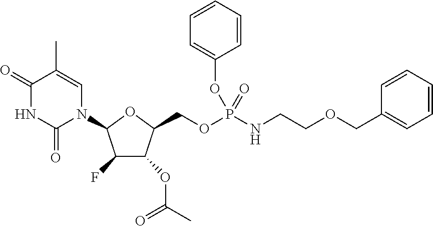 [SMILES: CC(=O)O[C@@H]1[C@@H](F)[C@@H](n2cc(C)c(=O)[nH]c2=O)O[C@H]1COP(=O)(NCCOCc1ccccc1)Oc1ccccc1]

Compound 111 can be prepared by acylation of Compound 110. [M−H]⁺ calculated for C₂₇H₃₁FN₃O₉P: 590.17.

### Example 12

### Ethyl 2-((((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzoate (Compound 112)

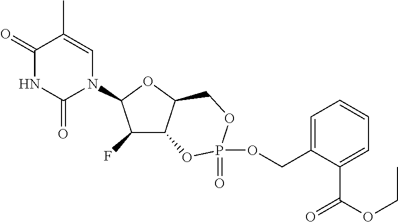

Compound 112 was prepared according to Scheme III from N,N,N′,N′-tetraisopropyl-1-(2-(1-ethyloxycarbonyl)benzyloxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a single isomer. [M−H]⁺ calculated for C₂₀H₂₂FN₂O₉P: 483.09; found: 483.1. ¹H NMR (400 MHz, CDCl₃) 8.25 (s, 1H), 7.95 (d, J 8.0, 1H), 7.62-7.45 (m, 4H), 7.09 (s, 1H), 6.42 (t, J=6.9, 1H), 5.56 (d, J=8.0, 2H), 5.40-5.20 (m, 1H), 4.70-4.52 (m, 2H), 4.42-4.32 (m, 3H), 3.91-3.88 (m, 1H), 1.98 (s, 3H), and 1.42 (t, J=9.6, 3H).

### Example 13

### Methyl 2-((((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzoate (Compound 113)

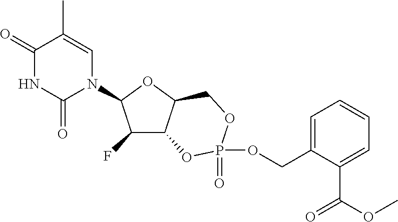

Compound 113 can be prepared according to Scheme III from N,N,N′,N′-tetraisopropyl-1-(2-(1-methyloxycarbonyl)benzyloxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₁₉H₂₀FN₂O₉P: 469.08.

### Example 14

### Propyl 2-((((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzoate (Compound 114)

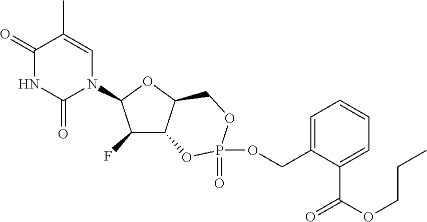

Compound 114 was prepared according to Scheme III from N,N,N′,N′-tetraisopropyl-1-(2-(1-propyloxycarbonyl)benzyloxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₁H₂₄FN₂O₉P: 497.11; found: 497.2.

### Example 15

### Isopropyl 2-((((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzoate (Compound 115)

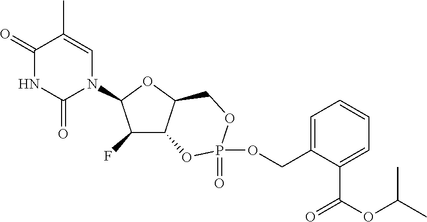

Compound 115 can be prepared according to Scheme III from N,N,N′,N′-tetraisopropyl-1-(2-(1-isopropyloxycarbonyl)benzyloxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₁H₂₄FN₂O₉P: 497.11.

### Example 16

### Butyl 2-((((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzoate (Compound 116)

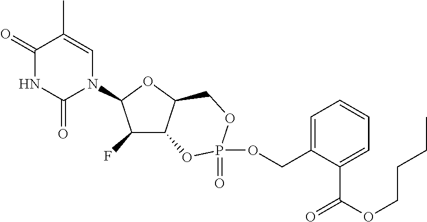

Compound 116 can be prepared according to Scheme III from N,N,N′,N′-tetraisopropyl-1-(2-(1-butyloxycarbonyl)benzyloxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₂H₂₆FN₂O₉P: 511.13.

### Example 17

### Isobutyl 2-((((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzoate (Compound 117)

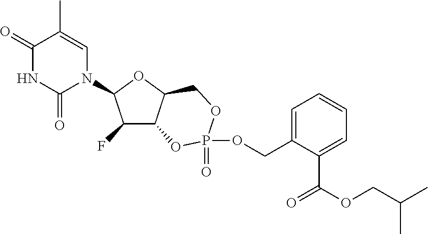

Compound 117 can be prepared according to Scheme III from N,N,N′,N′-tetraisopropyl-1-(2-(1-isobutyloxycarbonyl)benzyloxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₂H₂₆FN₂O₉P: 511.13.

### Example 18

### 1-((2S,3R,4S,5S)-5-(((4-(5-Chloro-2-fluorophenyl)-2-oxido-1,3,2-dioxaphosphinan-2-yl)oxy)methyl)-3-fluoro-4-hydroxytetrahydrofuran-2-yl)-5-methylpyrimidine-2,4(1H,3H)-dione (Compound 118)

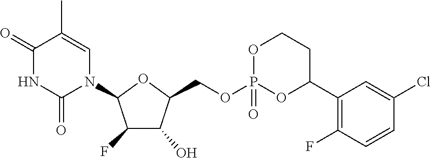 [SMILES: Cc1cn([C@H]2O[C@@H](COP3(=O)OCCC(c4cc(Cl)ccc4F)O3)[C@H](O)[C@H]2F)c(=O)[nH]c1=O]

Compound 118 can be prepared according to method from 1-(5-chloro-2-fluorophenyl)propane-1,3-diol and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₁₉H₂₀ClF₂N₂O₈P: 507.05.

### Example 19

### (2S,3S,4R,5S)-2-(((4-(3-Chlorophenyl)-2-oxido-1,3,2-dioxaphosphinan-2-yl)oxy)methyl)-4-fluoro-5-(5-methyl-2,4-dioxo-3,4-dihydropyrimidin-1(2H)-yl)tetrahydrofuran-3-yl acetate (Compound 119)

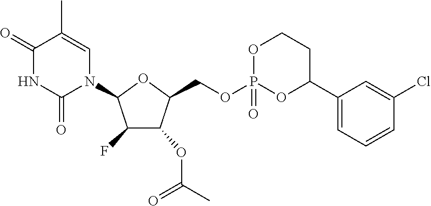 [SMILES: CC(=O)O[C@@H]1[C@@H](F)[C@@H](n2cc(C)c(=O)[nH]c2=O)O[C@H]1COP1(=O)OCCC(c2cccc(Cl)c2)O1]

Compound 119 can be prepared according to known literature method from 1-(5-chlorophenyl)propane-1,3-diol, acetyl chloride, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₁H₂₃ClFN₂O₉P: 531.07.

### Example 20

### 1-((4aS,6S,7R,7aS)-2-(((S)-1,1-Dipropoxypropan-2-yl)amino)-7-fluoro-2-oxidotetrahydro-4H-furo[3,2-d][1,3,2]dioxaphosphinin-6-yl)-5-methylpyrimidine-2,4(1H,3H)-dione (Compound 120)

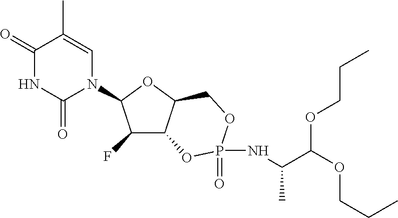

Compound 120 was prepared according to Scheme IV from substituted phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₉H₃₁FN₃O₈P: 478.17; Found: 478.2.

### Example 21

### Isobutyl ((4aS,6S,7R,7aS)-7-fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)-L-alaninate (Compound 121)

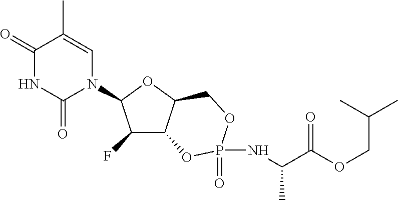

Compound 121 was prepared according to Scheme TV from substituted phenyl phosphorodichloridate, isobutyl L-alaninate, and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M+H]⁺ calculated for C₁₇H₂₅FN₃O₈P: 450.15; Found: 450.1.

### Example 22

### 1-((4aS,6S,7R,7aS)-7-Fluoro-2-((2-((4-methylbenzyl)oxy)ethyl)amino)-2-oxidotetrahydro-4H-furo[3,2-d][1,3,2]dioxaphosphinin-6-yl)-5-methylpyrimidine-2,4(1H,3H)-dione (Compound 122)

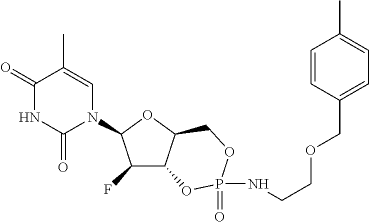

Compound 122 was prepared according to Scheme IV from substituted phenyl phosphorodichloridate, 2-((4-methylbenzyl)oxy)ethan-1-amine, and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M+H]⁺ calculated for C₂₀H₂₅FN₃O₇P: 470.15: Found: 470.1.

### Example 23

### 1-((4aS,6S,7R,7aS)-7-Fluoro-2-(heptyloxy)-2-oxidotetrahydro-4H-furo[3,2-d][1,3,2]dioxaphosphinin-6-yl)-5-methylpyrimidine-2,4(1H,3H)-dione (Compound 123)

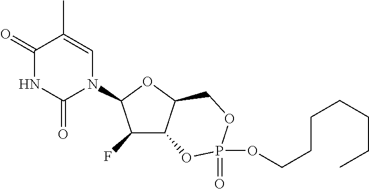

Compound 123 was prepared according to Scheme III from N,N,N′,N′-tetraisopropyl-1-heptyloxyphosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M+H]⁺ calculated for C₁₇H₂₆FN₂O₇P: 421.16: Found: 421.2.

### Example 24

### 1-((4aS,6S,7R,7aS)-7-Fluoro-2-(hexyloxy)-2-oxidotetrahydro-4H-furo[3,2-d][1,3,2]dioxaphosphinin-6-yl)-5-methylpyrimidine-2,4(1H,3H)-dione (Compound 124)

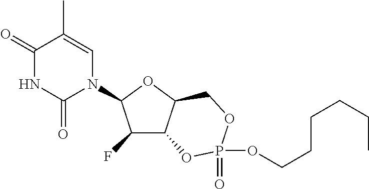

Compound 124 was prepared according to Scheme III from N,N,N′,N′-tetraisopropyl-1-hexyloxyphosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₆H₂₄FN₂O₇P: 405.12.

### Example 25

### 1-((4aS,6S,7R,7aS)-7-Fluoro-2-(octyloxy)-2-oxidotetrahydro-4H-furo[3,2-d][1,3,2]dioxaphosphinin-6-yl)-5-methylpyrimidine-2,4(1H,3H)-dione (Compound 125)

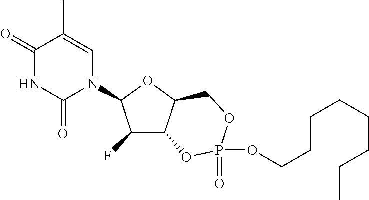

Compound 125 was prepared according to Scheme III from N,N,N′,N′-tetraisopropyl-1-octyloxyphosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₈H₂₈FN₂O₇P: 433.15.

### Example 26

### (2S,3S,4R,5S)-2-((((((S)-1,1-Dimethoxypropan-2-yl)amino)(phenoxy)phosphoryl)oxy)methyl)-4-fluoro-5-(5-methyl-2,4-dioxo-3,4-dihydropyrimidin-1(2H)-yl)tetrahydrofuran-3-yl propionate (Compound 126)

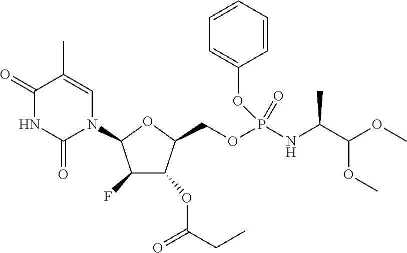 [SMILES: CCC(=O)O[C@@H]1[C@@H](F)[C@@H](n2cc(C)c(=O)[nH]c2=O)O[C@H]1COP(=O)(N[C@@H](C)C(OC)OC)Oc1ccccc1]

Compound 126 can be prepared by acylation of Compound 103. [M−H]⁺ calculated for C₂₄H₃₃FN₃O₁₀P: 572.18.

### Example 27

1-((4aS,6S,7R,7aS)-7-Fluoro-2-(benzyloxy)-2-oxidotetrahydro-4H-furo[3,2-d][1,3,2]dioxaphosphinin-6-yl)-5-methylpyrimidine-2,4(1H,3H)-dione (Compound 127)

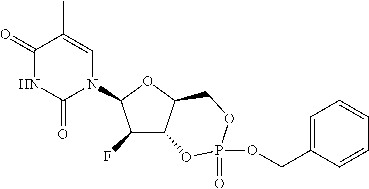

Compound 127 can be prepared according to Scheme III from N,N,N′,N′-tetraisopropyl-1-benzyloxyphosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₇H₁₈FN₂O₇P: 411.07.

### Example 28

### 1-((4aS,6S,7R,7aS)-7-Fluoro-2-((3-fluorobenzyl)oxy)-2-oxidotetrahydro-4H-furo[3,2-d][1,3,2]dioxaphosphinin-6-yl)-5-methylpyrimidine-2,4(1H,3H)-dione (Compound 128)

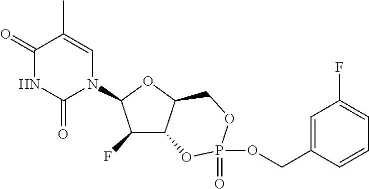

Compound 128 can be prepared according to Scheme III from N,N,N′,N′-tetraisopropyl-1-(3-fluorobenzyl)oxyphosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₇H₁₇F₂N₂O₇P: 429.06.

### Example 29

### 1-((4aS,6S,7R,7aS)-7-Fluoro-2-oxido-2-phenethoxytetrahydro-4H-furo[3,2-d][1,3,2]dioxaphosphinin-6-yl)-5-methylpyrimidine-2,4(1H,3H)-dione (Compound 129)

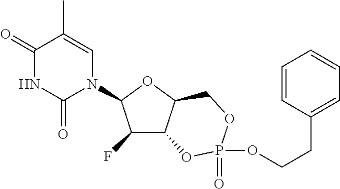

Compound 129 can be prepared according to Scheme III from N,N,N′,N′-tetraisopropyl-1-(2-phenethoxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₈H₂₀FN₂O₇P: 425.09.

### Example 30

### 1-((4aS,6S,7R,7aS)-7-Fluoro-2-oxido-2-(3-phenylpropoxy)tetrahydro-4H-furo[3,2-d][1,3,2]dioxaphosphinin-6-yl)-5-methylpyrimidine-2,4(1H,3H)-dione (Compound 130)

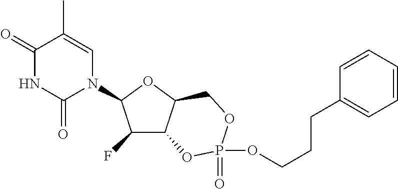

Compound 130 can be prepared according to Scheme III from N,N,N′,N′-tetraisopropyl-1-(3-phenylpropoxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₉H₂₂FN₂O₇P: 439.10.

### Example 31

### 2-((((4aS,6S, 7R,7aS)-7-Fluoro-6-(5-methyl-2,4-dioxo-3,4-dihydropyrimidin-1(2H)-yl)-2-oxidotetrahydro-4H-furo[3,2-d][1,3,2]dioxaphosphinin-2-yl)oxy)methyl)benzyl acetate (Compound 131)

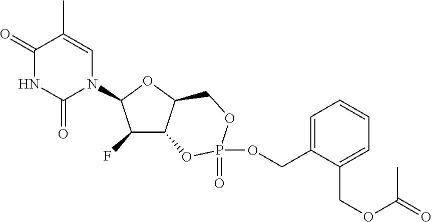

Compound 131 can be prepared according to Scheme III from 2-(((bis(diisopropylamino)phosphaneyl)oxy)methyl)benzyl acetate and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₂₀H₂₂FN₂O₉P: 483.09.

### Example 32

### 1-((4aS,6S,7R,7aS)-7-Fluoro-2-((2-(hydroxymethyl)benzyl)oxy)-2-oxidotetrahydro-4H-furo[3,2-d][1,3,2]dioxaphosphinin-6-yl)-5-methylpyrimidine-2,4(1H,3H)-dione (Compound 132

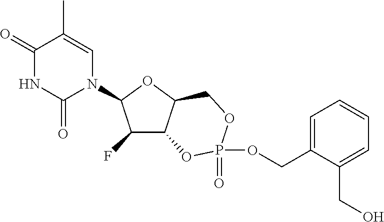

Compound 132 can be prepared according to Scheme III from 2-(((bis(diisopropylamino)phosphaneyl)oxy)methyl)benzyl acetate and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₈H₂₀FN₂O₈P: 441.08.

### Example 33

### 1-((4aS,6S,7R,7aS)-7-Fluoro-2-((2-(methoxymethyl)benzyl)oxy)-2-oxidotetrahydro-4H-furo[3,2-d][1,3,2]dioxaphosphinin-6-yl)-5-methylpyrimidine-2,4(1H,3H)-dione (Compound 133)

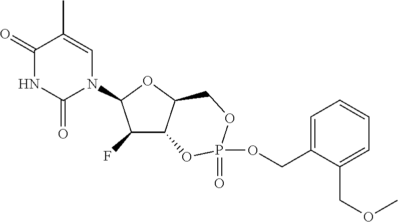

Compound 133 can be prepared according to Scheme III from N,N,N′,N′-tetraisopropyl-1-((2-(methoxymethyl)benzyl)oxy)phosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₁₉H₂₂FN₂O₈P: 455.10.

### Example 34

### 1-((4aS,6S,7R,7aS)-2-(((S)-1-(Benzyloxy)propan-2-yl)amino)-7-fluoro-2-oxidotetrahydro-4H-furo[3,2-d][1,3,2]dioxaphosphinin-6-yl)-5-methylpyrimidine-2,4(1H,3H)-dione (Compound 134)

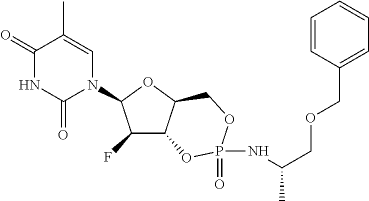

Compound 134 can be prepared according to Scheme 1V from substituted phenyl phosphorodichloridate, (S)-1-(benzyloxy)propan-2-amine, and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₂₀H₂₅FN₃O₇P: 468.13.

### Example 35

### 1-((4aS,6S,7R,7aS)-2-((2-(1,3-Dioxolan-2-yl)benzyl)oxy)-7-fluoro-2-oxidotetrahydro-4H-furo[3,2-d][1,3,2]dioxaphosphinin-6-yl)-5-methylpyrimidine-2,4(1H,3H)-dione (Compound 135)

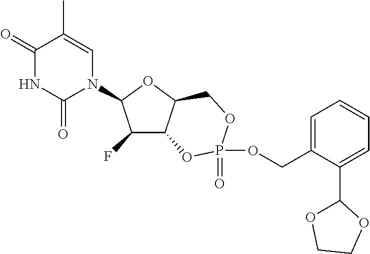

Compound 135 was prepared according to Scheme III from 1-((2-(1,3-dioxolan-2-yl)benzyl)oxy)-N,N,N′,N′-tetraisopropylphosphanediamine and 1-((2S,3R,4S,5S)-3-fluoro-4-hydroxy-5-(hydroxymethyl)tetrahydrofuran-2-yl)-5-methylpyrimidine-2,4(1H,3H)-dione as a mixture of two diastereomers. [M−H]⁺ calculated for C₂₀H₂₂FN₂O₉P: 483.09; Found: 482.9.

### Example 36

### 1-((4aS,6S,7R,7aS)-2-(((S)-1,1-Dibutoxypropan-2-yl)amino)-7-fluoro-2-oxidotetrahydro-4H-furo[3,2-d][1,3,2]dioxaphosphinin-6-yl)-5-methylpyrimidine-2,4(1H,3H)-dione (Compound 136)

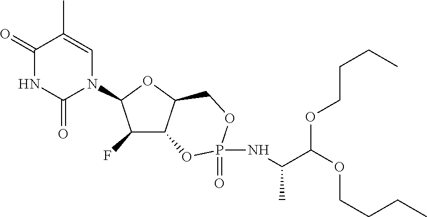

Compound 136 can be prepared according to Scheme IV from substituted phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₂₁H₃₅FN₃O₈P: 478.17.

### Example 37

### 1-((4aS,6S,7R,7aS)-2-(((S)-1-(1,3-Dioxolan-2-yl)ethyl)amino)-7-fluoro-2-oxidotetrahydro-4H-furo[3,2-d][1,3,2]dioxaphosphinin-6-yl)-5-methylpyrimidine-2,4(1H,3H)-dione (Compound 13

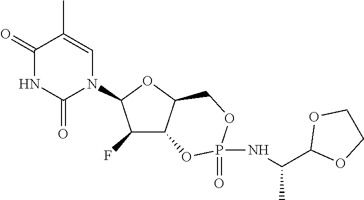

Compound 137 can be prepared according to Scheme IV from substituted phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₁₅H₂₁FN₃O₈P: 420.10.

### Example 38

### 1-((4aS,6S,7R,7aS)-7-Fluoro-2-oxido-2-(((S)-1-(4,4,5,5-tetramethyl-1,3-dioxolan-2-yl)ethyl)amino)tetrahydro-4H-furo[3,2-d][1,3,2]dioxaphosphinin-6-yl)-5-methylpyrimidine-2,4(1H,3H)-dione (Compound 138)

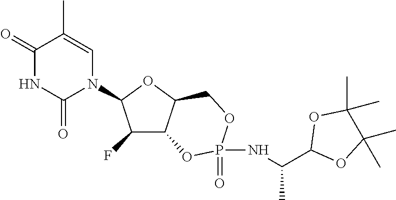

Compound 138 can be prepared according to Scheme IV from substituted phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₁₉H₂₉FN₃O₈P: 476.16.

### Example 39

### 1-((4aS,6S,7R,7aS)-2-(((S)-1-(1,3-Dioxan-2-yl)ethyl)amino)-7-fluoro-2-oxidotetrahydro-4H-furo[3,2-d][1,3,2]dioxaphosphinin-6-yl)-5-methylpyrimidine-2,4(1H,3H)-dione (Compound 139)

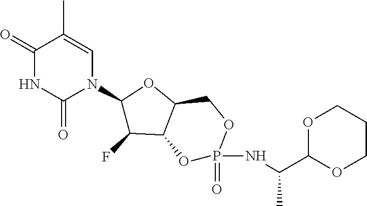

Compound 139 can be prepared according to Scheme 1V from substituted phenyl phosphorodichloridate, (S)—N-Cbz-2-aminopropanol, and 1-((2S,3R,4S,5S)-3-fluoro-4-hydroxy-5-(hydroxymethyl)tetrahydrofuran-2-yl)-5-methylpyrimidine-2,4(1H,3H)-dione. [M−H]⁺ calculated for C₁₆H₂₃FN₃O₈P: 434.11.

### Biological Examples

Examples of use of the method include the following. It will be understood that the following are examples and that the method is not limited solely to these examples. Example A: Tissue Distribution Following Oral Administration of reference compounds and the disclosed compounds

The liver specificity of the disclosed compounds is compared relative to a corresponding active compound in liver and other organs that could be targets of toxicity.

### Methods:

Reference compounds and the phosphoramidate and cyclophosphate compounds are administered at 5-20 mg/kg to fasted rats by oral gavage. Plasma concentrations of the active, metabolite, and phosphoramidate and cyclophosphate compounds in circulation and in the hepatic portal vein are determined by HPLC-UV, and the liver, small intestine, and other organ concentrations are measured by LC-MS using the standard chromatography method.

Table 1 provides the results of reference and selected new compounds, which demonstrates the liver targeting of the phosphoramidate and cyclophosphate compounds and provide evidence for improved efficiency of the compounds over other types of compounds in liver-targeting and/or achieving high level of the active in the liver. This can occur solely by the high efficiency liver targeting provided by the new compounds.

TABLE 1
Drug and metabolite levels in the liver and blood 5 hour after oral
administration of selected compounds at 5 mg/kg dose in rats

| | NUCₗᵢᵥₑᵣ | NUC_{blood} | NTPₗᵢᵥₑᵣ | NTPₗᵢᵥₑᵣ/ |
|---|---|---|---|---|
| Compound | (ng/g) | (ng/mL) | (ng/g) | NUC_{blood} |
| AI-2173 | 47 | 20.4 | 21 | 1.0 |
| 105 | 34.1 | 23 | 55.1 | 2.4 |
| 112 | 244 | 118 | 118 | 1.0 |
| 121 | 60.9 | 38.7 | 168 | 4.3 |
| 122 | 32.7 | 14.1 | 66.7 | 4.7 |

NUCₗᵢᵥₑᵣ—The nucleoside level in the liver;
NUC_{blood}—The nucleoside level in blood;
NTPₗᵢᵥₑᵣ—The nucleoside triphosphate level in the liver

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15%, 10%, 5%, 3%, 1$, 0.1%, or otherwise. Similarly, in certain embodiments, the terms “generally perpendicular” and “substantially perpendicular” refer to a value, amount, or characteristic that departs from exactly perpendicular by less than or equal to 15%, 10%, 5%, 3%, 1%, 0.1%, or otherwise.

The above description discloses several methods and materials. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it covers all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Although the invention has been described with reference to embodiments and examples, it should be understood that numerous and various modifications can be made without departing from the spirit of the invention. Accordingly, the invention is limited only by the following claims.

What is claimed is:

1. A compound of Formula I, II, III, IV, V or VI, or VII: 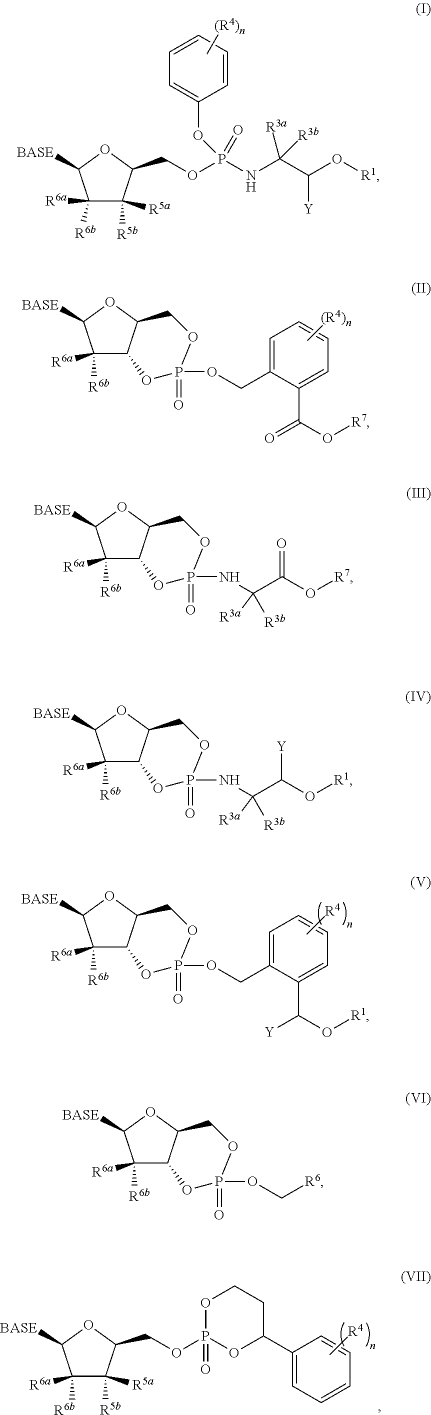
or a stereoisomer or a pharmaceutically acceptable salt thereof,
wherein:
Y is H or —OR²;
R¹ and R² are independently H, an optionally substituted C₁-C₁₀ alkyl, an optionally substituted C₅-C₁₀ aryl, an optionally substituted C₁-C₆ alkyl-C(O)—, an optionally substituted five to ten membered heteroaryl, or an optionally substituted —(CH₂)ₓ—(C₅-C₁₀ aryl); or alternatively, R¹ and R² may be taken together with the atoms to which they are attached and intervening atoms to form a four to ten membered heterocycle optionally substituted with between one and four C₁-C₆ alkyl groups,
R^{3a} and R^{3b} are independently H or an optionally substituted C₁-C₁₀ alkyl; or
alternatively, R^{3a} and R^{3b} may be taken together with the atom to which they are attached form a three to seven membered ring,
R⁴ is halo, an optionally substituted C₁-C₁₀ alkyl, or an optionally substituted C₁-C₆ alkyloxy;
R^{5a}, R^{5b}, R^{6a}, and R^{6b} are independently selected from the group consisting of H, OR^{A}, halo, —CN, and an optionally substituted C₁-C₁₀ alkyl;
R^{A} is selected from the group consisting of H, an optionally substituted C₁-C₆ acyl, an optionally substituted C₁-C₆ alkyloxycarbonyl; and an optionally substituted C₁-C₆ carbamoyl;
R⁷ is H or an optionally substituted C₁-C₁₀ alkyl;
R⁸ is selected from the group consisting of an optionally substituted C₁-C₁₅ alkyl, an optionally substituted aryl, an optionally substituted benzyl, an optionally substituted phenylethyl, and an optionally substituted phenylpropyl.
n is 0, 1, 2, or 3;
x is 0, 1, or 2; and
Base is a natural nucleoside base or a derivative or analog thereof.

2. The compound of claim 1, wherein the compound is of Formula (I):

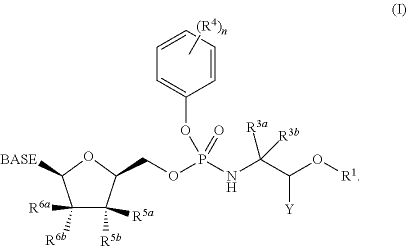

3. The compound of claim 1, wherein the compound is of Formula (IV):

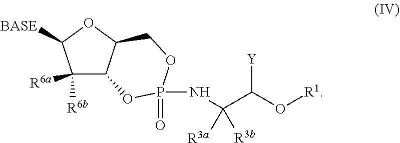

4. The compound of claim 1, wherein the compound is of Formula (V):

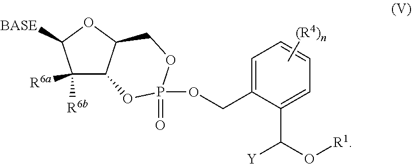

5. The compound of any one of claims 1-4, wherein R¹ is a C₁-C₆ alkyl.

6. The compound of any one of claims 1-5, wherein R¹ is methyl.

7. The compound of any one of claims 1-5, wherein R¹ is ethyl.

8. The compound of any one of claims 1-5, wherein R¹ is propyl.

9. The compound of any one of claims 1-5, wherein R¹ is butyl.

10. The compound of any one of claims 1-4, wherein R¹ is H.

11. The compound of any one of claims 1-4, wherein aryl is phenyl.

12. The compound of any one of claims 1-4 and 11, wherein x is 1.

13. The compound of any one of claims 1-12, wherein Y is H.

14. The compound of any one of claims 1-12, wherein Y is —OR².

15. The compound of claim 14, wherein R² is methyl.

16. The compound of claim 14, wherein R² is ethyl.

17. The compound of claim 14, wherein R² is propyl.

18. The compound of claim 14, wherein R² is butyl.

19. The compound of claim 1, wherein R¹ and R² are taken together with the atoms to which they are attached and intervening atoms to form a four to ten membered heterocycle optionally substituted with between one and four C₁-C₆ alkyl groups.

20. The compound of claim 1, wherein R¹ and R² are taken together with the atoms to which they are attached and intervening atoms to form

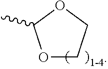

21. The compound of claim 20, wherein R¹ and R² are taken together with the atoms to which they are attached and intervening atoms to form

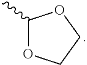

22. The compound of claim 20, wherein R¹ and R² are taken together with the atoms to which they are attached and intervening atoms to form

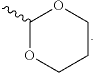

23. The compound of claim 1, wherein the compound is of Formula (VI):

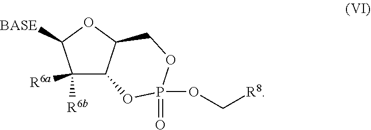

24. The compound of claim 1 or 23, wherein R⁸ is C₁-C₁₅ alkyl.

25. The compound of any one of claims 1, 23, and 24, wherein R⁸ is pentyl.

26. The compound of any one of claims 1, 23, and 24, wherein R⁸ is hexyl.

27. The compound of any one of claims 1, 23, and 24, wherein R⁸ is heptyl.

28. The compound of any one of claims 1, 23, and 24, wherein R⁸ is phenyl.

29. The compound of any one of claims 1, 23, and 24, wherein R⁸ is fluorophenyl.

30. The compound of any one of claims 1, 23, and 24, wherein R⁸ is benzyl.

31. The compound of any one of claims 1, 23, and 24, wherein R⁸ is phenylethyl.

32. The compound of claim 1, wherein the compound is of Formula (III):

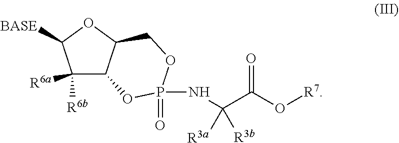

33. The compound of any one of claims 1-3, 5-22, and 32, wherein R^{3a} and R^{3b} are both H.

34. The compound of any one of claims 1-3, 5-22, and 32, wherein R^{3a} is H and R^{3b} is methyl.

35. The compound of claim 1, wherein the compound is of Formula (VII):

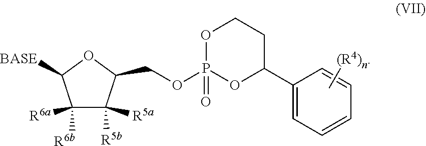

36. The compound of any one of claims 1-, 2, 5-22, and 35, wherein R^{5a} is H.

37. The compound of any one of claims 1-, 2, 5-22, 35, and 36, wherein R^{5b} is —OR^{A}.

38. The compound of any one of claims 1-, 2, 5-22, and 35-37, wherein R^{A} is H.

39. The compound of any one of claims 1-, 2, 5-22, and 35-37, wherein R^{5b} is

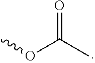

40. The compound of any one of claims 1-, 2, 5-22, and 35-37, wherein R^{5b} is

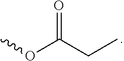

41. The compound of claim 1, wherein the compound is of Formula (II):

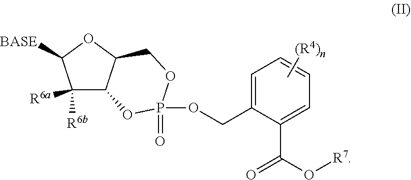

42. The compound of claim 1 or 41, wherein R⁷ is C₁-C₆ alkyl.

43. The compound of any one of claims 1, 41, and 42, wherein R⁷ is methyl.

44. The compound of any one of claims 1, 41, and 42, wherein R⁷ is ethyl.

45. The compound of any one of claims 1, 41, and 42, wherein R⁷ is propyl.

46. The compound of any one of claims 1, 41, and 42, wherein R⁷ is i-Pr.

47. The compound of any one of claims 1, 41, and 42, wherein R⁷ is butyl.

48. The compound of any one of claims 1, 41, and 42, wherein R⁷ is i-butyl.

49. The compound of any one of claims 1-48, wherein R^{6a} is halo.

50. The compound of any one of claims 1-49, wherein R^{6a} is fluoro.

51. The compound of any one of claims 1-50, wherein R^{6b} is H.

52. The compound of any one of claims 1, 2, 4-22, and 33-51, wherein R⁴ is halo selected from fluoro and chloro.

53. The compound of any one of claims 1-, 2, 4-22, and 33-52, wherein n is 1.

54. The compound of any one of claims 1, 2, 4-22, and 33-52, wherein n is 2.

55. The compound of any one of claims 1, 2, 4-22, 33-52 and 54, wherein one R⁴ is fluoro and one R⁴ is chloro.

56. The compound of any one of claims 1-55, wherein Base is selected from the group consisting of 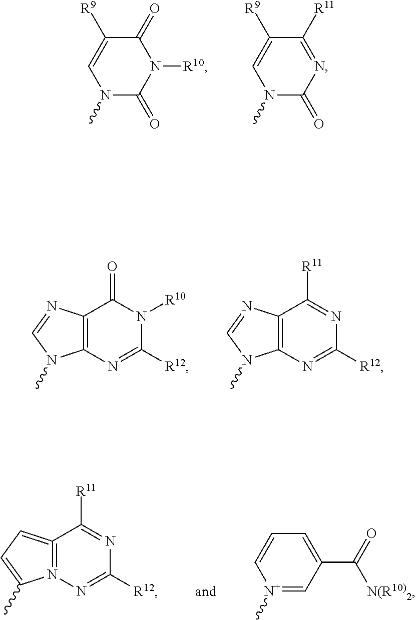
wherein:
R⁹ is H, halo, —CD₃, or an optionally substituted C₁-C₁₀ alkyl;
R¹⁰ is selected from the group consisting of H, an optionally substituted C₁-C₁₀ alkyl, an optionally substituted C₁-C₁₀ alkyl-OCH₂—, an optionally substituted C₁-C₁₀ alkyl-NHCH₂—, an optionally substituted C₁-C₁₀ acyl, an optionally substituted C₁-C₁₀ alkyl-OC(O)—, an optionally substituted (C₆₋₁₀ aryl)-CH₂OCH₂—, an optionally substituted (C₆₋₁₀ aryl)-OCH₂—, an optionally substituted (C₆₋₁₀ aryl)-C(O)—, and an optionally substituted (C₆₋₁₀ aryl)-OC(O)—;
R¹¹ is selected from the group consisting of OH, NH₂, NHOH, an optionally substituted C₁-C₁₀ alkyloxy, an optionally substituted C₁-C₁₀ alkylamino, an optionally substituted C₁-C₁₀ acyloxy, an optionally substituted C₁-C₁₀ acylamino, an optionally substituted C₁-C₁₀ alkyl-OC(O)NH—, an optionally substituted (C₆₋₁₀ aryl)-C(O)O—, an optionally substituted (C₆₋₁₀ aryl)-C(O)NH—, an optionally substituted (C₆₋₁₀ aryl)-OC(O)NH—, an optionally substituted C₁-C₁₀ alkyl-OCH₂NH—, and an optionally substituted C₁-C₁₀ alkyl-OCH₂O—; and
R¹² is selected from a group of H, NH₂, an optionally substituted C₁-C₁₀ alkylamino, an optionally substituted C₁-C₁₀ acylamino, an optionally substituted C₁-C₁₀ alkyl-OC(O)NH—, an optionally substituted (C₆₋₁₀ aryl)-C(O)NH—, an optionally substituted (C₆₋₁₀ aryl)-OC(O)NH—, and an optionally substituted C₁-C₁₀ alkyl-OCH₂NH—.

57. The compound of any one of claims 1-56, wherein Base is

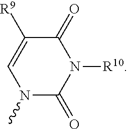

58. The compound of any one of claims 1-57, wherein R⁹ is methyl.

59. The compound of any one of claims 1-58, wherein R¹⁰ is H.

60. A compound selected from the group consisting of: 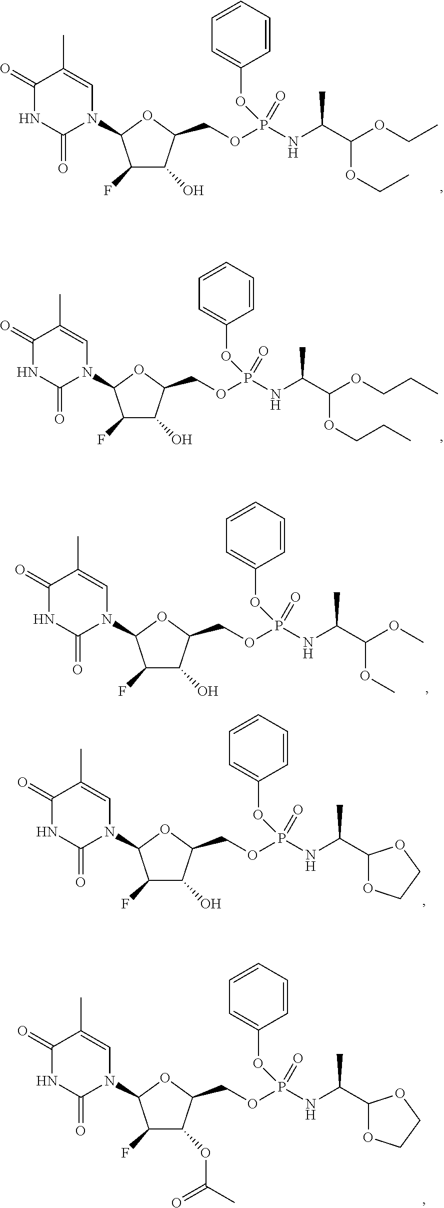 [SMILES: CCOC(OCC)[C@H](C)NP(=O)(OC[C@@H]1O[C@H](n2cc(C)c(=O)[nH]c2=O)[C@H](F)[C@H]1O)Oc1ccccc1 ; CCCOC(OCCC)[C@H](C)NP(=O)(OC[C@@H]1O[C@H](n2cc(C)c(=O)[nH]c2=O)[C@H](F)[C@H]1O)Oc1ccccc1 ; COC(OC)[C@H](C)NP(=O)(OC[C@@H]1O[C@H](n2cc(C)c(=O)[nH]c2=O)[C@H](F)[C@H]1O)Oc1ccccc1 ; Cc1cn([C@H]2O[C@@H](COP(=O)(N[C@@H](C)C3OCCO3)Oc3ccccc3)[C@H](O)[C@H]2F)c(=O)[nH]c1=O ; CC(=O)O[C@@H]1[C@@H](F)[C@@H](n2cc(C)c(=O)[nH]c2=O)O[C@H]1COP(=O)(N[C@@H](C)C1OCCO1)Oc1ccccc1] 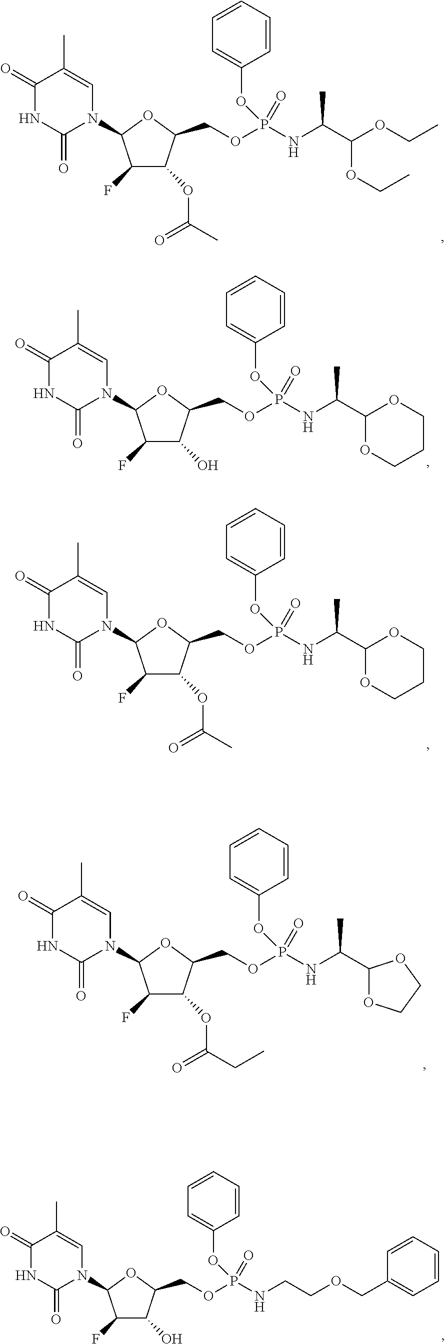 [SMILES: CCOC(OCC)[C@H](C)NP(=O)(OC[C@@H]1O[C@H](n2cc(C)c(=O)[nH]c2=O)[C@H](F)[C@H]1OC(C)=O)Oc1ccccc1 ; Cc1cn([C@H]2O[C@@H](COP(=O)(N[C@@H](C)C3OCCCO3)Oc3ccccc3)[C@H](O)[C@H]2F)c(=O)[nH]c1=O ; CC(=O)O[C@@H]1[C@@H](F)[C@@H](n2cc(C)c(=O)[nH]c2=O)O[C@H]1COP(=O)(N[C@@H](C)C1OCCCO1)Oc1ccccc1 ; CCC(=O)O[C@@H]1[C@@H](F)[C@@H](n2cc(C)c(=O)[nH]c2=O)O[C@H]1COP(=O)(N[C@@H](C)C1OCCO1)Oc1ccccc1 ; Cc1cn([C@H]2O[C@@H](COP(=O)(NCCOCc3ccccc3)Oc3ccccc3)[C@H](O)[C@H]2F)c(=O)[nH]c1=O] 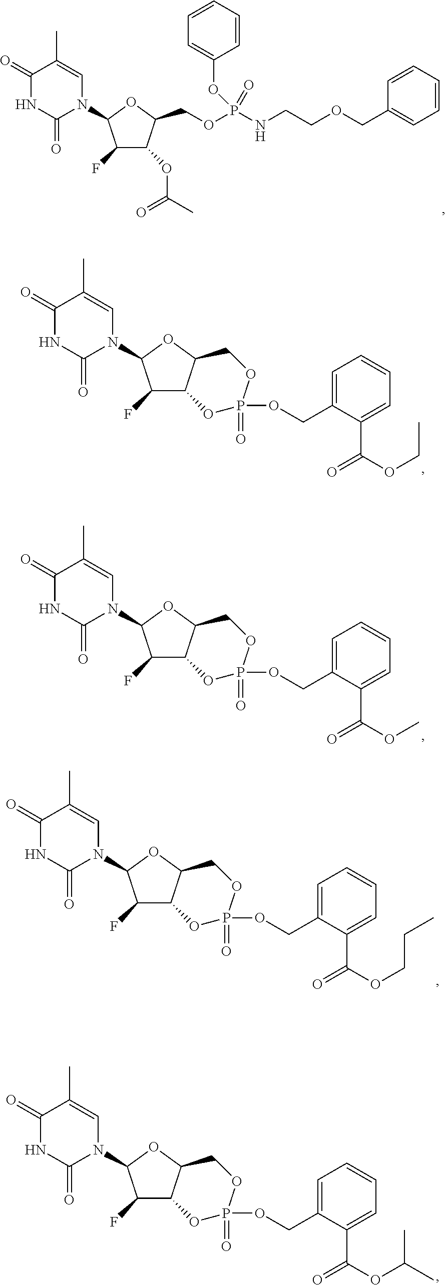 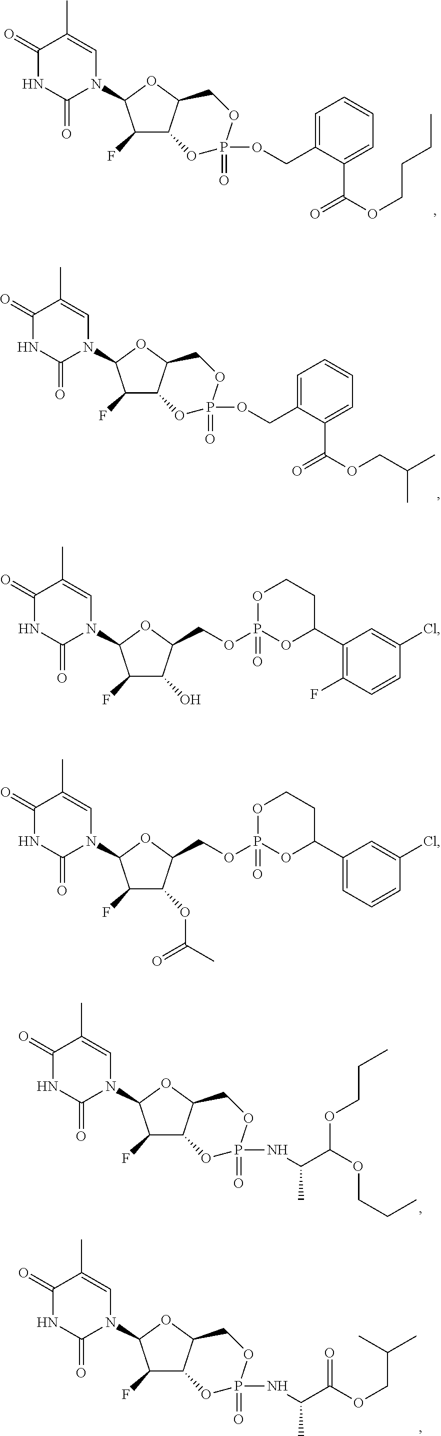 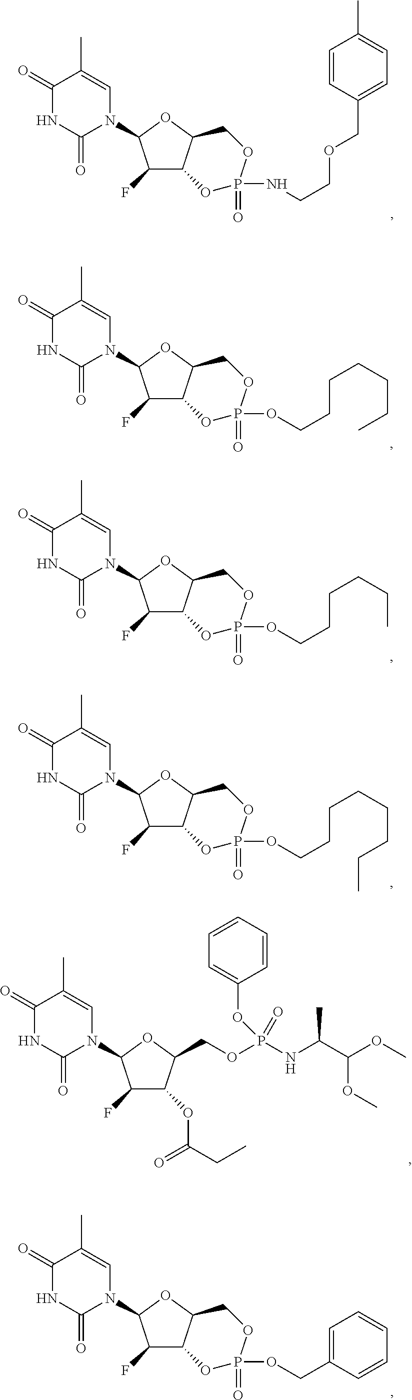 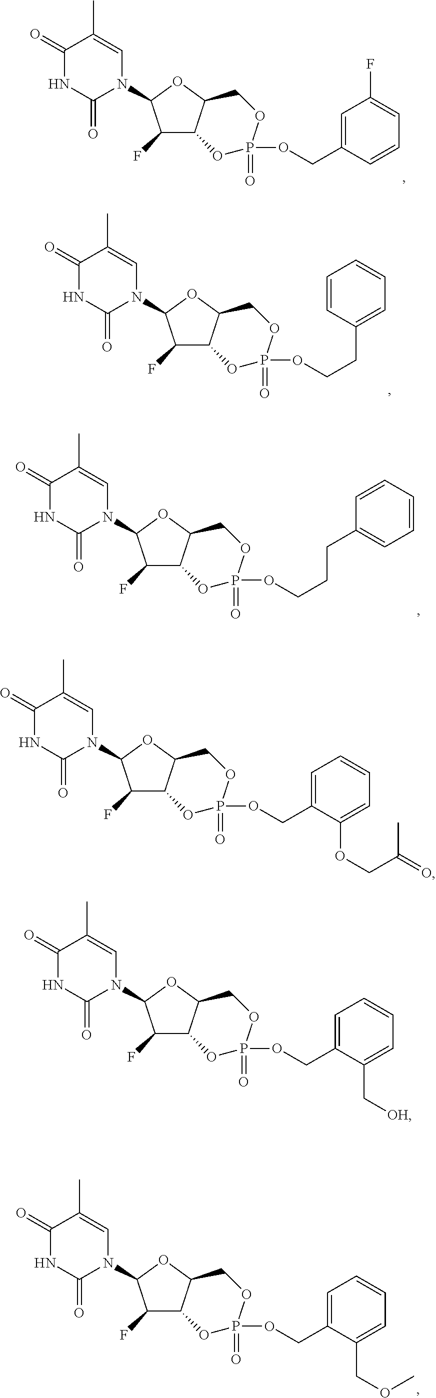 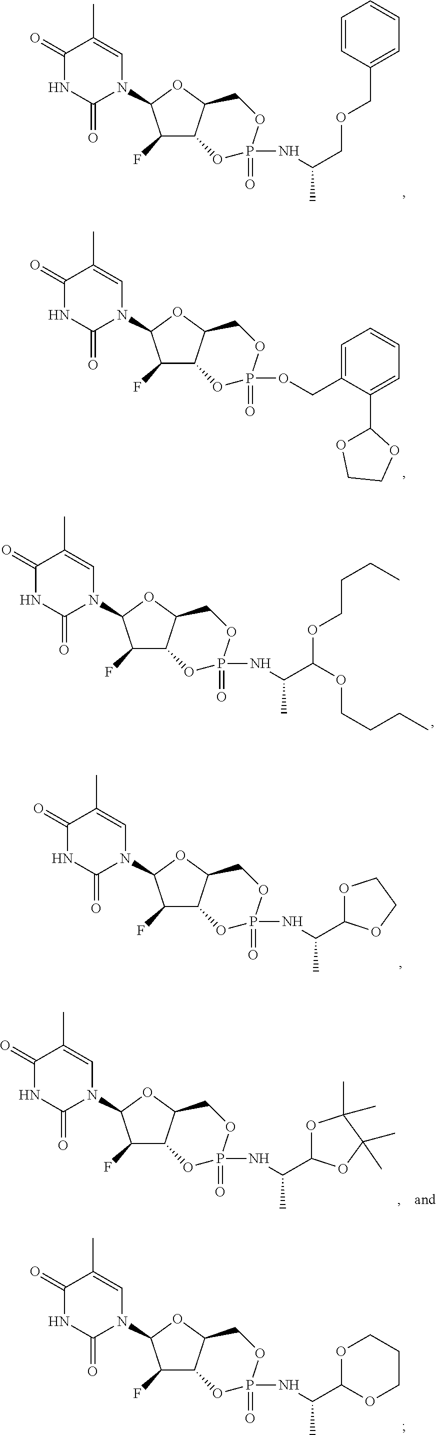
or a stereoisomer or a pharmaceutically acceptable salt thereof.

61. A pharmaceutical composition comprising the compound of any one of claims 1-60 and a pharmaceutically acceptable excipient.

62. The pharmaceutical composition of claim 61, further comprising one or more anti-cancer agents.

63. A method of treating a disease, disorder or condition comprising administering an effective amount of the compound of any one of claims 1-60 to a subject in need thereof.

64. The method of claim 63, wherein the disease, disorder, or condition is a disease, disorder, or condition of the liver.

65. The method of claim 63, wherein the disease, disorder or condition is a metabolic, cardiovascular, or hormonal disease in which the liver is involved in the production and/or the homeostasis control of the biochemical end products of the disease, disorder, or condition.

66. The method of claim 63, wherein the disease, disorder or condition is selected from the group consisting of hepatitis, cancer, liver fibrosis, fatty liver, malaria, viral infection, parasitic infection, diabetes, hyperlipidemia, atherosclerosis, obesity, dyslipidemia, hyperglycemia, and a hormonal condition.

67. The method of claim 63, wherein the disease, disorder or condition is a viral infection, cancer, or other disease in which the prodrug compounds enhance the distribution of an active drug to the target tissue or cell.

68. The method of any one of claims 63-67, wherein the subject is a mammal.

69. The method of any one of claims 63-68, wherein the subject is a human.

70. The method of any one of claims 63-69, further comprising administering an effective amount of at least one additional therapeutic agent to the subject in need thereof.

71. The method of claim 70, wherein the additional therapeutic agent is one or more of sorafenib, regorafenib, an immune-oncology agent such as a PD-1 or PD-L1 checkpoint inhibitor.

72. A compound of any one of claims 1-60 for use in treating a disease or condition in the liver or a disease or condition in which the physiological or pathogenic pathways involve the liver.

73. Use of a compound of any one of claims 1-60 in the preparation of a medicament for treating a disease or condition in the liver or a disease or condition in which the physiological or pathogenic pathways involve the liver.
